(19) United States
(12) Patent Application Publication
(10) Pub. No.: US 2026/0137802 A1
(43) Pub. Date: May 21, 2026

(54) CD117-TARGETING NANOPARTICLES FOR USE IN DRUG DELIVERY

(71) Applicant: CRISPR THERAPEUTICS AG, Zug (CH)

(72) Inventor: Megan D. WARNER, Cambridge, MA (US)

(21) Appl. No.: 18/877,012

(22) PCT Filed: Jun. 20, 2023
(86) PCT No.: PCT/IB2023/056371

§ 371 Date: Dec. 19, 2024

Related U.S. Application Data
(60) Provisional application No. 63/353,734, filed on Jun. 20, 2022.
(60) Provisional application No. 63/398,033, filed on Aug. 15, 2022.

(51) Int. Cl. A61K 47/69; A61K 47/68; C07K 16/28; C12N 9/22; C12N 15/11; C12N 15/88

(52) U.S. Cl. CPC A61K 47/6913; A61K 47/6849; C07K 16/2803; C12N 9/222; C12N 15/11; C12N 15/88; C07K 2317/565; C07K 2317/567; C07K 2317/569; C07K 2319/01; C12N 2310/20

(57) ABSTRACT

Single domain antibody fragments binding to CD117 and lipid nanoparticle conjugates associated with the antibodies and cargos of interest (e.g., a gene editing system). Also provided herein are methods of delivering the cargos to CD117⁺ cells using the lipid nanoparticle conjugates and methods of preparing such lipid nanoparticle conjugates.

### CROSS-REFERENCE TO RELATED APPLICATIONS

This application claims the benefit of U.S. Provisional Application No. 63/353,734, filed Jun. 20, 2022, and U.S. Provisional Application No. 63/398,033, filed Aug. 15, 2022, the content of each of which are herein incorporated by reference in their entirety.

### SEQUENCE LISTING STATEMENT

The contents of the electronic sequence listing titled CRISP_41833_601.xml (Size: 123,334 bytes; and Date of Creation: Jun. 16, 2023) is herein incorporated by reference in its entirety.

### BACKGROUND OF THE INVENTION

Cluster of differentiation 117 (CD117), also known as tyrosine-protein kinase (cKIT) or mast/stem cell growth factor receptor (SCFR), is a cytokine receptor expressed on hematopoietic stem cells and on other types of cells. Upon binding to stem cell factor (SCF), the tyrosine kinase activity of CD117 is activated, leading to the phosphorylation and activation of downstream molecules. This CD117-mediated cell signaling plays roles in various biological processes, including cell survival, proliferation, and differentiation.

CD117 is an important cell surface marker of hematopoietic cells. For example, hematopoietic stem cells (HSCs), multipotent progenitors, and common myeloid progenitor cells all express high levels of CD117. Accordingly, CD117 can be used as a target for diagnosis of and drug delivery to such hematopoietic cells.

### SUMMARY OF THE INVENTION

The present disclosure is based, at least in part, on the development of single domain antibodies (e.g., VHH) having binding activity to human CD117 and optionally to non-human primate CD117. Certain anti-CD117 VHH antibodies disclosed herein (e.g., P10 and P38) show high binding activity to CD117⁺ cells without inhibiting ligand-induced cKIT phosphorylation. SCF was found not to interfere with binding of such anti-CD117 antibodies to CD117. These features indicate that the anti-CD117 VHH antibodies disclosed herein are suitable candidates for use in guiding delivery of cargos (e.g., gene editing systems) to CD117⁺ cells with less impact on cell activity.

Accordingly, provided herein are single domain antibodies capable of binding to CD117 (e.g., human CD117) and vehicles (e.g., lipid nanoparticles) carrying such for use in drug delivery to CD117⁺ cells. Also provided herein are nucleic acids comprising nucleotide sequences encoding the anti-CD117 antibodies, vectors and host cells carrying such, and methods for producing the antibodies, as well as methods for preparing lipid nanoparticle (LNP) conjugates comprising the anti-CD117 antibodies.

In some aspects, the present disclosure features an antibody that binds CD117, comprising a single domain antibody fragment, which comprises:
(i) a complementarity determining region 1 (CDR1) set forth as GX₁X₂TX₃X₄X₅X₆X₇ (SEQ ID NO: 2), in which X₁ is D, G, H, R, or T; X₂ is T or absent; X₃ is F, L, S, or V; X₄ is G, S, or T; X₅ is I, N, S, T or Y; X₆ is D, V, or Y; and X₇ is A, F, P, S, V or W;
(ii) a complementarity determining region 2 (CDR2) set forth as X₁X₂X₃X₄X₅X₆X₇X₈ (SEQ ID NO: 39), in which X₁ is I or V; X₂ is A, G, H, L, R, S, T, or V, X₃ is R, S, or W; X₄ is G, N, S, or Y; X₅ is A, G, L, or absent; X₆ is A, D, G, L, or S; X₇ is G, M, S, T, or V; and X₈ is A, L, or T; and
(iii) a complementarity determining region 3 (CDR3) set forth as: (a) GRFHPIRVDTA (SEQ ID NO: 69); (b) ASGSNWRLGAIDEY (SEQ ID NO: 71); (c) GQHLSGLGGSAWSIEG (SEQ ID NO: 73); (d) RQYVGSGSYYLKKEGGY (SEQ ID NO: 75); (e) DSTGVYGTGYVSSRKGRY (SEQ ID NO: 77); (f) AFTPEFRDGGIWDDASV (SEQ ID NO: 79); (g) VRRRWLIWQEEEY (SEQ ID NO: 83); (h) DQRGVPAYYSDYALY (SEQ ID NO: 85); (i) DESFPAYYSDYALY (SEQ ID NO: 86); (j) VLRTGM (SEQ ID NO: 67); (k) SDSYFYASPHLY (SEQ ID NO: 80); (l) SDTYFYASPHLY (SEQ ID NO: 81); or (m) RRGTILVVQEYEY (SEQ ID NO: 84).

In some embodiments, the CDR3 in the anti-CD117 antibody is set forth as any one of (a)-(i) listed above. In some examples, the CDR3 is AFTPEFRDGGIWDDASV (SEQ ID NO: 79). In some examples, the CDR3 is DQRGVPAYYSDYALY (SEQ ID NO: 85). In some examples, the CDR3 is DESFPAYYSDYALY (SEQ ID NO: 86).

In some embodiments, X₁ in CDR1 can be D, G, H, or R (e.g., R); X₂ in CDR1 can be absent; X₃ in CDR1 can be F, L, or S (e.g., F or S); X₄ in CDR1 can be G, S, or T; X₅ in CDR1 can be S or Y; X₆ in CDR1 can be D or Y; and X₇ in CDR1 can be A or V. In some examples, the CDR1 of the anti-CD117 antibody can be one of the following: (a) GRTTFSTYW (SEQ ID NO: 8); (b) GGTFSIYP (SEQ ID NO: 11); (c) GRTLSNYF (SEQ ID NO: 14); (d) GRTFSSYA (SEQ ID NO: 17); (e) GHTFSNYA (SEQ ID NO: 20); (f) GDTFSSYS (SEQ ID NO: 30); (g) GRTSGSYV (SEQ ID NO: 36); and (h) GRTFTYDA (SEQ ID NO: 37). In one example, the CDR1 is GRTFSSYA (SEQ ID NO: 17). In another example, the CDR1 is GRTSGSYV (SEQ ID NO: 36). In yet another example, the CDR1 is GRTFTYDA (SEQ ID NO: 37).

In some embodiments, X₁ in the CDR2 of the anti-CD117 antibody can be I; X₂ in the CDR2 can be G, H, L, R, S, or T (e.g., L, or S); X₃ in the CDR2 can be S or W; X₄ in the CDR2 can be N, S, or Y (e.g., N or S); X₅ in the CDR2 can be A, G, or L (e.g., A or G); X₆ in the CDR2 can be G, L, or S; X₇ in the CDR2 can be G, M, T, or V (e.g., M, S, or T); and X₈ in the CDR2 can be is A or T (e.g., T). In some examples, the CDR2 can be one of the following: (a) ISWSAGMA (SEQ ID NO: 43); (b) IGWSASGT (SEQ ID NO: 45); (c) IHWSLGST (SEQ ID NO: 47); (d) ITSSGLVA (SEQ ID NO: 49); (e) ISWSGGST (SEQ ID NO: 51); (f) ILSNGLTT (SEQ ID NO: 53); (g) IRWSGGTT (SEQ ID NO: 59); and (h) ISWSAGMT (SEQ ID NO: 63). In one example, the CDR2 can be ILSNGLTT (SEQ ID NO: 53). In another example, the CDR2 can be ISWSAGMT (SEQ ID NO: 63). In yet another example, the CDR2 can be ISWSGGST (SEQ ID NO: 51).

In specific examples, the single domain antibody fragment comprises the same CDR1, same CDR2, and same CDR3 as a reference antibody of P10, P12, P27, P29, P31, P32, P35, P37, or P38. In one example, the single domain antibody fragment comprises the same CDR1, same CDR2, and same CDR3 as those of P10. In another example, the single domain antibody fragment comprises the same CDR1, same CDR2, and same CDR3 as those of P31. In yet another example, the single domain antibody fragment comprises the same CDR1, same CDR2, and same CDR3 as those of P38.

Any of the single domain antibody fragments disclosed herein can be a heavy chain variable domain antibody (VHH). In some embodiments, the single domain antibody fragment has the structure of, from N-terminus to C-terminus, framework (FR) 1 (FR1)-CDR1-FR2-CDR2-FR3-CDR3-FR4, and wherein:
(a) the FR1 is set forth as X₁VQLVESGGGLVX₂AGX₃SLRLSCX₄X₅S (SEQ ID NO: 1), in which X₁ is E or Q; X₂ is Q or R; X₃ is G or D; X₄ is A, T, or V; and X₅ is A, G, or V;
(b) the FR2 is set forth as X₁X₂WX₃RQX₄PGKX₅REX₆VX₇X₈ (SEQ ID NO: 3), in which X₁ is L, M, R, or V; X₂ is A, G, or H; X₃ is F, L, or Y; X₄ is A or G; X₅ is E, N, Q, or R; X₆ is F or L; X₇ is A, G, or S; and X₈ is A, G or S;
(c) the FR3 is set forth as X₁YX₂DSX₃X₄GRFTISRDX₅X₆X₇X₈TVYLX₉MX₁₀SLKPEDTAZX₁₁YYCAA (SEQ ID NO: 40), in which X₁ is L, N, or Y; X₂ is A, G, L, P, or Q; X₃ is M, V, or absent; X₄ is E or K; X₅ is G, K, or N; X₆ is A, G, T, or V; X₇ is E, K, or R; X₈ is D, N, or S; X₉ is H, Q or R; X₁₀ is D, N, or S; and X₁₁ is N, T, or V; and
(d) the FR4 is set forth as X₁X₂WX₃QGTX₄VTVSS (SEQ ID NO: 66) in which X₁ is D, E, L, R, or T; X₂ is D, S, or Y; X₃ is G or A; and X₄ is L or Q.

In some examples, the FR1 may comprise one of the following:

(SEQ ID NO: 7)
(a) EVQLVESGGGLVRAGGSLRLSCAAS;
(SEQ ID NO: 10)
(b) QVQLVESGGGLVQAGGSLRLSCAAS;
(SEQ ID NO: 13)
(c) QVQLVESGGGLVQAGDSLRLSCAVS;
(SEQ ID NO: 16)
(d) QVQLVESGGGLVQAGDSLRLSCAAS;
(SEQ ID NO: 19)
(e) QVHLVESGGGLVQAGGSLGLSCAAS;
(SEQ ID NO: 22)
(f) QVQLVESGGGLVQAGGSLRLSCVAS;
(SEQ ID NO: 29)
(g) QVQLVESGGGLVQAGGSLRLSCTAS;
and
(SEQ ID NO: 35)
(h) EVQLVESGGGLVQAGGSLRLSCAAS.

Alternatively or in addition, the FR2 may comprise one or the following:

(SEQ ID NO: 9)
(a) LGWFRQAPGKNREFVAA;
(SEQ ID NO: 12)
(b) VGWFRQAPGKQREFVAA;
(SEQ ID NO: 15)
(c) MAWLRQAPGKEREFVAA;
(SEQ ID NO: 18)
(d) MGWFRQAPGKEREFVAS;
(SEQ ID NO: 21)
(e) MGWFRQAPGKEREFVAA;
(SEQ ID NO: 23)
(f) MGWFRQGPGKEREFAAA;
(SEQ ID NO: 31)
(g) MGWFRQGPGKEREFVGG;
or
(SEQ ID NO: 38)
(h) RAWFRQAPGKEREFVAA.

Alternatively or in addition, the FR3 may comprise one of the following:

(SEQ ID NO: 44)
(a) YYQDSKGRFTISRDNTKNTAYLQMNSLQPEDTAVYYCAA;
(SEQ ID NO: 46)
(b) YYGDSVEGRFTVSRDNARSTVYLRMSSLKPDDTAVYYCAA;
(SEQ ID NO: 48)
(c) YYQDPVKGRFTISRDKAKNTVYLQMNTLKPEDTATYICAA;
(SEQ ID NO: 50)
(d) YYGDSVEARFTISRDNAKNTVYLQMDSLKPEDTAVYYCAA;
(SEQ ID NO: 52)
(e) LYADSVKGRFTISRDNGENTVYLQMNSLKPEDTAVYYCAL;
(SEQ ID NO: 54)
(f) YYADSVKGRFTISRDNAKDTVYLQMNSLKPEDTANYYCAA;
(SEQ ID NO: 60)
(g) YYADSVKGRFTISRDNAKNTVYLQMNSLKREDTAVYYCAA;
(SEQ ID NO: 56)
(h) YYADSVKGRFTISRDNAKNTVYLQMNSLKPEDTAVYYCAA
or
(SEQ ID NO: 65)
(i) YYPNSMKGRFTISRDNAKNTVYLQMNSLKPEDTAVYYCAA.

Alternatively or in addition, the FR4 may comprise one of the following:

(SEQ ID NO: 70)
(a) LYWAQGTQVTVSS;
(SEQ ID NO: 72)
(b) DSWGQGTQVTVSS;
(SEQ ID NO: 74)
(c) DYWGQGTQVTVSS;
(SEQ ID NO: 76)
(d) EYWGPGTQVTVSS;
(SEQ ID NO: 78)
(e) EYWGQGTLVTVSS;
(SEQ ID NO 68)
(f) DYWGQGTLVTVSS;
or
(SEQ ID NO: 87)
(g) RYWGQGTLVTVSS.

In some examples, the single domain antibody fragment has the same FR1, same FR2, same FR3, and same FR4 as a reference antibody of P10, P12, P27, P29, P31, P32, P35, P37, or P38 (e.g., P10, P31, or P38). In specific examples, the single domain antibody fragment comprises P10, P12, P27, P29, P31, P32, P35, P37, or P38. In one example, the single domain antibody fragment is P10. In another example, the single domain antibody fragment is P31. In yet another example, the single domain antibody fragment is P38.

In some embodiments, the single domain antibody fragment as disclosed herein may comprise a sortase recognition motif at the C-terminus (e.g., comprising the LPXTG (SEQ ID NO: 88) motif, in which X can be any amino acid residue). In one specific example, the sortase recognition motif is LPETGG (SEQ ID NO: 89). In some examples, the single domain antibody fragment may comprise a motif of LPXTGGGK (SEQ ID NO: 90) at the C-terminus. In one example, the C-terminal motif may be LPETGGGK (SEQ ID NO: 91).

Any of the anti-CD117 antibodies disclosed herein may comprise a functional group conjugated to the single domain antibody fragment (e.g., to the C-terminus of the antibody or to one or more internal amino acid residue, e.g., one or more lysine residues). Such a functional group would allow conjugation of the anti-CD117 antibody to a vehicle such as those disclosed herein via forming a covalent bond between the functional group and the vehicle. Examples of the functional groups include, but are not limited to, an azide group, a dibenzocyclooctyne group (DBCO), biotin, streptavidin, or a thiol group.

In other embodiments, the anti-CD117 antibody disclosed herein may further comprise an Fc fragment, which is linked to the C-terminus of the single domain antibody fragment. In some instances, the antibody may be a monovalent antibody. Alternatively, the antibody may be a bivalent antibody.

In other aspects, provided herein is a lipid nanoparticle conjugate, comprising (a) a lipid nanoparticle (LNP), and (b) an antibody that binds CD117 (anti-CD117 antibody), which can be any of the anti-CD117 antibodies as disclosed herein. The anti-CD117 antibody is attached on the surface of the LNP. In some instances, the anti-CD17 antibody is linked to a polyethylene glycol (PEG) moiety (e.g., PEG2000), which can be conjugated (e.g., via covalent bonds) to a lipid molecule (e.g., a PEG-lipid) contained in the LNP. For example, the anti-CD17 antibody may contain a first functional group (e.g., those disclosed herein such as an azide group) at the C-terminus and is covalently linked to the PEG moiety modified by second functional group via a reaction between the first and second functional groups to form a covalent bond.

Any of the lipid nanoparticle conjugates disclosed herein may further comprise a cargo, which can be encapsulated by or attached to the LNP. In some instances, the cargo may be a therapeutic agent or a diagnostic agent. In some examples, the cargo may be a gene editing system, which comprises a nuclease or a nucleic acid encoding the nuclease. In some instances, the nuclease can be an RNA-guided nuclease and the gene editing system further comprises a guide RNA (gRNA) or a nucleic acid encoding the gRNA. For example, the RNA-guided nuclease is a Cas9 enzyme, which optionally can be a Streptococcus pyogenes Cas9 enzyme.

The present disclosure also provides a method for delivering an agent to cells, the method comprising: contacting a lipid nanoparticle conjugate as disclosed herein with cells expressing CD117 (CD117⁺ cells) to allow for delivery of the cargo contained in the lipid nanoparticle conjugate to the CD117⁺ cells. In some embodiments, the CD117⁺ cells may comprise hematopoietic cells. In some embodiments, the contacting step is performed by administering the lipid nanoparticle conjugate to a subject in need thereof to deliver the agent to the CD117⁺ cells in the subject.

In addition, provided herein is a method for editing a gene in a CD117⁺ cell, comprising: contacting a lipid nanoparticle conjugate as disclosed herein with a cell expressing CD117 (CD117⁺ cell) to allow for delivery of the cargo contained in the lipid nanoparticle conjugate to the CD117⁺ cell. The cargo is a gene editing system, which edits a target gene in the CD117⁺ cell. In some examples, the CD117⁺ cells comprise hematopoietic cells. In some examples, the contacting step is performed by administering the lipid nanoparticle conjugate to a subject in need thereof to deliver the gene editing system to the CD117⁺ cell in the subject. In some instances, the subject is a human patient having a genetic disease associated with the target gene.

In some aspects, the present disclosure also provides a method for preparing a lipid nanoparticle conjugate, the method comprising: contacting an anti-CD117 antibody as disclosed herein with a plurality of LNPs to allow for attachment of the anti-CD117 antibody to the surface of the LNPs, thereby producing lipid nanoparticle conjugate(s).

In some embodiments, the anti-CD117 antibody is linked to a PEG moiety conjugated to a lipid molecule (PEG-lipid molecule). In some examples, the anti-CD117 antibody linked to the PEG-lipid molecule is prepared by a process comprising: (a) providing the anti-CD117 antibody, which comprises a sortase recognition motif at the C-terminus; (b) incubating the anti-CD117 antibody with a sortase peptide substrate in the presence of a sortase enzyme, which catalyzes a sortase reaction to conjugate the sortase peptide substrate to the sortase recognition motif (e.g., cleavage between the T and G residues found in the LPETGG (SEQ ID NO: 89) motif), wherein the sortase peptide substrate comprises a GGGK (SEQ ID NO: 93) motif (e.g., an azide functional group); thereby producing a functionalized anti-CD117 antibody having the functional group linked to the C-terminus residue; and (c) contacting the functionalized anti-CD117 antibody with the PEG-lipid molecule to allow for formation of a covalent bond between the functionalized anti-CD117 antibody and the PEG-lipid molecule, which can be modified by a second functional group such as DBCO, thereby producing the anti-CD117 antibody linked to the PEG-lipid molecule via a reaction between the first and second functional groups to form a covalent bond. In some embodiments, the sortase recognition motif comprises LPXTG (SEQ ID NO: 88) and produces the fragment LPXT (SEQ ID NO: 94) upon sortage cleavage. The C-terminus T residue can be linked to the sortase peptide substrate upon the sortase-mediated transpeptidation reaction.

In addition, the present disclosure provides a nucleic acid comprising a nucleotide sequence encoding an anti-CD117 antibody as disclosed herein, a vector (e.g., an expression vector) comprising the nucleic acid, and host cells comprising such a vector. Further, the present disclosure provides a method for producing the anti-CD117 antibody disclosed herein, the method comprising: (a) culturing the host cell of claim 32 to allow for expressing of the anti-CD117 antibody; and (b) harvesting the anti-CD117 antibody thus produced.

Also within the scope of the present disclosure are cargo-loaded delivery vehicles as disclosed herein (e.g., LNPs conjugated, covalently or non-covalently, directly or indirectly, to any of the anti-CD117 antibodies) for use in delivering the cargo to CD117⁺ cells such as hematopoietic stem cells and thus for therapeutic or diagnostic purposes. When the cargo is a gene editing system, the delivery vehicles disclosed herein are for use in genetic editing of a target gene in the CD117⁺ cells and for treating a genetic disease associated with the target gene.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A and 1B are diagrams depicting VHH antibodies in monovalent form (FIG. 1A) and bivalent form (FIG. 1B).

FIGS. 2A and 2B include diagrams showing delivery efficiencies of CD117-targeting VHH-LNPs carrying an mRNA encoding green fluorescent protein (GFP). FIG. 2A:
hematopoietic stem cells (HSCs). FIG. 2B: Kasumi-1 cells.

FIG. 3 includes a diagram showing gene editing efficiency in Kasumi-1 cells using CD117-targeting VHH-LNPs carrying a CRISPR/Cas9-mediated gene editing system.

FIGS. 4A-4C include diagrams illustrating sortase-mediated reaction for functionalize anti-CD117 antibodies. FIG. 4A: a diagram illustrating Sortase A site-specifically modifies a range of molecules. FIG. 4B: a diagram illustrating Sortase A-mediated transpeptidation reaction for modifying an anti-CD117 VHH antibody. FIG. 4C: a diagram showing sortase-mediated conjucation of a functional group (azide as an example) to an anti-CD117 VHH antibody.

### DETAILED DESCRIPTION OF THE INVENTION

Gene editing is a promising approach for treating diseases associated with genetic mutations, e.g., by knocking out a disease-causing gene or by repairing genetic mutations involved in the disease. In vivo gene editing requires delivering target tissue/cells a gene editing system, which typically comprises an endonuclease and optionally a guide RNA. It is challenging to efficiently and accurately deliver the gene editing system to specific target cells, given the large size of endonucleases commonly used in gene editing and the requirement of delivering multiple components simultaneously into the target cells.

The present disclosure is based, at least in part, on the development of efficient and target cell-specific drug delivery vehicles, which can be used to deliver cargos, including gene editing systems, to specific target cells such as hematopoietic cells. The drug delivery vehicles comprise an antibody such as a single domain antibody (e.g., VHH) that specifically binds CD117 (anti-CD117 antibody), which is a surface marker for various types of hematopoietic cells. The anti-CD117 antibody can be attached to the surface of a vehicle, such as an LNP, which can encapsulate or be associated with a cargo (e.g., components of a gene editing system) to be delivered to the target cells. Thus, provided herein are anti-CD117 antibodies, delivery vehicles such as LNP conjugates comprising the anti-CD117 antibodies, methods of using such in cargo delivery, and methods for making the delivery vehicles or the anti-CD117 antibodies.

As used herein, the term “conjugate” refers to a chemical association between two substances, for example, by covalent attachment or by non-covalent attachment. In some instances, the two entities may be conjugated directly. In other instances, the two entities may be conjugated via a linker (indirectly), which can be of any suitable type.

### I. Anti-CD117 Antibodies

CD117, also known as cKIT, is encoded by the proto-oncogene c-KIT. As a cell surface receptor, CD117 has an extracellular domain, a transmembrane domain, and a cytoplasmic protein kinase domain. Structures of CD117 from various origins are known in the art. As one example, the structure information of human CD117 is reported under Gene ID: 3815 and the amino acid sequence of human CD117 can be found under GenBank accession number AAH71593.1.

In some aspects, the present disclosure provides antibodies that bind CD117 (e.g., human CD117). Such anti-CD117 antibodies as disclosed herein may cross-react with both human CD117 and non-human primate CD117 (e.g., cynomolgus CD117). In some embodiments, the anti-CD117 antibodies provided herein are single domain antibodies.

### A. Single Domain Anti-CD17 Antibodies

Single-domain antibodies, also known as nanobodies, are small antigen-binding fragments containing only one heavy or light chain variable region (as opposed to conventional antibodies having both heavy and light chain variable regions). In some instances, the single domain antibodies provided herein are heavy chain only antibodies (VHH antibodies) containing a single heavy chain variable region.

Like conventional antibodies, a single domain antibody such as a VHH antibody, contains regions of hypervariability, also known as “complementarity determining regions” (“CDR”), interspersed with regions that are more conserved, which are known as “framework regions” (“FR”). A VHH antibody is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

In some embodiments, an antibody moiety disclosed herein may share the same complementary determining regions (CDRs) as a reference antibody. Two antibodies having the same CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/).

In some embodiments, an antibody moiety disclosed herein may share a certain level of sequence identity as compared with a reference sequence. The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25 (17): 3389-3402, 1997. When utilizing BLAST and Gapped BLAST programs, the default parameters of the respective programs (e.g., XBLAST and NBLAST) can be used.

In some embodiments, an antibody moiety disclosed herein may have one or more amino acid variations relative to a reference antibody. The amino acid residue variations as disclosed in the present disclosure (e.g., in framework regions and/or in CDRs) can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-CD117 single domain antibody disclosed herein comprises the consensus sequence of each of FR1-CDR1-FR2-CDR2-FR3-CDR3-FR4 listed in Table 1 below. Exemplary sequences of each of these domains in an anti-CD117 antibody as disclosed herein are also provided in Table 1. The anti-CD117 antibody provided herein may contain one or more such sequences.

A sequence alignment of exemplary anti-CD117 VHH antibodies is provided in Example 1 below. All of these exemplary anti-CD117 VHH antibodies are within the scope of the present disclosure.

In some examples, the anti-CD117 antibody is one of the exemplary antibodies (reference antibodies) provided in Table 1 and in the sequence alignment in Example 1 below. For example, the anti-CD117 may be P10, P12, P27, P29, P31, P32, P35, P37, or P38. In one example, the anti-CD117 antibody is P10. In another example, the anti-CD117 antibody is P38. In yet another example, the anti-CD117 antibody is P31.

In other examples, the anti-CD117 antibody disclosed herein may comprise amino acid variations in one or more of the framework regions relative to the corresponding framework regions in a reference antibody as disclosed herein (e.g., P10, P12, P27, P29, P31, P32, P35, P37, or P38). For example, the anti-CD117 antibody may comprise, collectively, up to 15 amino acid variations (e.g., up to 12, 10, 9, 8, 7, 6, 5, 4, 3, 2, or 1 amino acid variations) in one or more framework regions relative to the corresponding framework regions in the reference antibody.

In some instances, the anti-CD117 antibody may comprise up to 8 amino acid variations (e.g., up to 7, 6, 5, 4, 3, 2, or 1 amino acid variations) in one or more of the CDRs collectively relative to those in the CDRs of a reference antibody (e.g., P10, P12, P27, P29, P31, P32, P35, P37, or P38). In some instances, the anti-CD117 moiety may comprise the same CDR3 as the CDR3 of the reference antibody and comprise one or more amino acid variations (e.g., up to 5, 4, 3, 2, or 1) in one or more of the other CDRs.

Any of the anti-CD117 antibodies disclosed herein (e.g., one of the example antibodies such as P10, P12, P27, P29, P31, P32, P35, P37, or P38, e.g., P10, P31, and P38) can be used for making the drug delivery vehicle disclosed herein.

TABLE 1
Amino Acid Sequences of Exemplary Anti-CD117 VHH Antibodies

| | FR1 | CDR1 | FR2 |
|---|---|---|---|
| Consensus Sequence | X₁VQLVESGGGLVX₂AGX₃SLRLSCX₄ | GX₁X₂TX₃X₄X₅X₆X₇ (SEQ ID | X₁X₂WX₃RQX₄PGKX₅REX₆VX₇X₈ (SEQ |
| | X₅S (SEQ ID NO: 1) | NO: 2) | ID NO: 3) |
| MP16 | EVQLVESGGGLVQAGGSLRLSCVAS | GTTSGIVA (SEQ ID NO: 5) | MHWYRQAPGKRRELVAS (SEQ ID NO: |
| | (SEQ ID NO: 4) | | 6) |
| P-0027 | EVQLVESGGGLVRAGGSLRLSCAAS | GRTTFSTYW (SEQ ID NO: 8) | LGWFRQAPGKNREFVAA (SEQ ID NO: |
| | (SEQ ID NO: 7) | | 9) |
| P-0035 | QVQLVESGGGLVQAGGSLRLSCAAS | GGTFSIYP (SEQ ID NO: 11) | VGWFRQAPGKQREFVAA (SEQ ID NO: |
| | (SEQ ID NO: 10) | | 12) |
| P-0037 | QVQLVESGGGLVQAGDSLRLSCAVS | GRTLSNYF (SEQ ID NO: 14) | MAWLRQAPGKEREFVAA (SEQ ID NO: |
| | (SEQ ID NO: 13) | | 15) |
| P-0032 | QVQLVESGGGLVQAGDSLRLSCAAS | GRTFSSYA (SEQ ID NO: 17) | MGWFRQAPGKEREFVAS (SEQ ID NO: |
| | (SEQ ID NO: 16) | | 18) |
| P-0029 | QVHLVESGGGLVQAGGSLGLSCAAS | GHTFSNYA (SEQ ID NO: 20) | MGWFRQAPGKEREFVAA (SEQ ID NO: |
| | (SEQ ID NO: 19) | | 21) |
| P-0010 | QVQLVESGGGLVQAGGSLRLSCVAS | GRTFSSYA (SEQ ID NO: 17) | MGWFRQGPGKEREFAAA (SEQ ID NO: |
| | (SEQ ID NO: 22) | | 23) |
| MP14 | QVQLVESGGGLVQAGGSLRLSCAGS | GRTVSTYA (SEQ ID NO: 25) | MGWFRQAPGKEREFVSA (SEQ ID NO: |
| | (SEQ ID NO: 24) | | 26) |
| MP6 | QVQLVESGGGLVQAGGSLRLSCVGS | GRTVSTYA (SEQ ID NO: 25) | MGWFRQAPGKERELVAA (SEQ ID NO: |
| | (SEQ ID NO: 27) | | 28) |
| P-0012 | QVQLVESGGGLVQAGGSLRLSCTAS | GDTFSSYS (SEQ ID NO: 30) | MGWFRQGPGKEREFVGG (SEQ ID NO: |
| | (SEQ ID NO: 29) | | 31) |
| MP7 | QVQLVESGGGLVQAGGSLRLSCAAS | GRTFSSYV (SEQ ID NO: 33) | MGWFRQAPGKEREFVAA (SEQ ID NO: |
| | (SEQ ID NO: 10) | | 21) |
| P-0038 | EVQLVESGGGLVQAGGSLRLSCAAS | GRTSGSYV (SEQ ID NO: 36) | MGWFRQAPGKEREFVAA (SEQ ID NO: |
| | (SEQ ID NO: 35) | | 21) |
| P-0031 | EVQLVESGGGLVRAGGSLRLSCAAS | GRTFTYDA (SEQ ID NO: 37) | RAWFRQAPGKEREFVAA (SEQ ID NO: |
| | (SEQ ID NO: 7) | | 38) |
| | CDR2 | FR3 | |
| Consensus Sequence | X₁X₂X₃X₄X₅X₆X₇X₈ (SEQ ID NO: | X₁YX₂DSX₃X₄GRFTISRDX₅X₆X₇X₈TVYLX₉MX₁₀SLKPEDTAZX₁₁YYCAA (SEQ | |
| | 39) | ID NO: 40) | |
| MP16 | VVRGASL (SEQ ID NO: 41) | NYLDSVKGRFIISEDGARNTVYLQMNSLKPEDTAVYYCNA (SEQ ID NO: 42) | |
| P-0027 | ISWSAGMA (SEQ ID NO: 43) | YYQDSKGRFTISRDNTKNTAYLQMNSLQPEDTAVYYCAA (SEQ ID NO: 44) | |
| P-0035 | IGWSASGT (SEQ ID NO: 45) | YYGDSVEGRFTVSRDNARSTVYLRMSSLKPDDTAVYYCAA (SEQ ID NO: 46) | |
| P-0037 | IHWSLGST (SEQ ID NO: 47) | YYQDPVKGRFTISRDKAKNTVYLQMNTLKPEDTATYICAA (SEQ ID NO: 48) | |
| P-0032 | ITSSGLVA (SEQ ID NO: 49) | YYGDSVEARFTISRDNAKNTVYLQMDSLKPEDTAVYYCAA (SEQ ID NO: 50) | |
| P-0029 | ISWSGGST (SEQ ID NO: 51) | LYADSVKGRFTISRDNGENTVYLQMNSLKPEDTAVYYCAL (SEQ ID NO: 52) | |
| P-0010 | ILSNGLTT (SEQ ID NO: 53) | YYADSVKGRFTISRDNAKDTVYLQMNSLKPEDTANYYCAA (SEQ ID NO: 54) | |
| MP14 | IARSGGGT (SEQ ID NO: 55) | YYADSVKGRFTISRDNAKNTVYLQMNSLKPEDTAVYYCAA (SEQ ID NO: 56) | |
| MP6 | IGRSGGST (SEQ ID NO: 57) | YYADSVKGRFTISRDNVRNTVYLQMNSLKPEDAAVYYCAA (SEQ ID NO: 58) | |
| P-0012 | IRWSGGTT (SEQ ID NO: 59) | YYADSVKGRFTISRDNAKNTVYLQMNSLKREDTAVYYCAA (SEQ ID NO: 60) | |
| MP7 | ISWYGDST (SEQ ID NO: 61) | YYADSVKGRFTISRDNAKNTVYLHMNSLKPEDTAVYYCAA (SEQ ID NO: 62) | |
| P-0038 | ISWSAGMT (SEQ ID NO: 63) | YYADSVKGRFTISRDNAKNTVYLQMNSLKPEDTAVYYCAA (SEQ ID NO: 56) | |
| P-0031 | ISWSGGST (SEQ ID NO: 51) | YYPNSMKGRFTISRDNAKNTVYLQMNSLKPEDTAVYYCAA (SEQ ID NO: 65) | |
| | CDR3 | FR4 | |
| Consensus Sequence | | X₁X₂WX₃QGTX₄VTVSS (SEQ ID | |
| | | NO: 66) | |
| MP16 | VLRTGM (SEQ ID NO: 67) | DYWGQGTLVTVSS (SEQ ID NO: | |
| | | 68) | |
| P-0027 | GRFHPIRVDTA (SEQ ID NO: | LYWAQGTQVTVSS (SEQ ID | |
| | 69) | NO: 70) | |
| P-0035 | ASGSNWRLGAIDEY (SEQ ID NO: | DSWGQGTQVTVSS (SEQ ID | |
| | 71) | NO: 72) | |
| P-0037 | GQHLSGLGGSAWSIEG (SEQ ID | DYWGQGTQVTVSS (SEQ ID NO: | |
| | NO: 73) | 74) | |
| P-0032 | RQYVGSGSYYLKKEGGY (SEQ ID | EYWGPGTQVTVSS (SEQ ID | |
| | NO: 75) | NO: 76) | |
| P-0029 | DSTGVYGTGYVSSRKGRY (SEQ ID | EYWGQGTLVTVSS (SEQ ID | |
| | NO: 77) | NO: 78) | |
| P-0010 | AFTPEFRDGGIWDDASV (SEQ ID | DYWGQGTQVTVSS (SEQ ID NO: | |
| | NO: 79) | 74) | |
| MP14 | SDSYFYASPHLY (SEQ ID NO: | DYWGQGTLVTVSS (SEQ ID NO: | |
| | 80) | 68) | |
| MP6 | SDTYFYASPHLY (SEQ ID NO: | TDWGQGTQVTVSS (SEQ ID | |
| | 81) | NO: 82) | |
| P-0012 | VRRRWLIWQEEEY (SEQ ID NO: | DYWGQGTLVTVSS (SEQ ID NO: | |
| | 83) | 68) | |
| MP7 | RRGTILVVQEYEY (SEQ ID NO: | DYWGQGTLVTVSS (SEQ ID NO: | |
| | 84) | 68 | |
| P-0038 | DQRGVPAYYSDYALY (SEQ ID | DYWGQGTQVTVSS (SEQ ID NO: | |
| | NO: 85) | 74) | |
| P-0031 | DESFPAYYSDYALY (SEQ ID NO: | RYWGQGTLVTVSS (SEQ ID | |
| | 86) | NO: 87) | |

* In consensus sequence of CDR1: X₁ is D, G, H, R, or T; X₂ is T or absent; X₃ is F, L, S, or V; X₄ is G, S, or T; X₅ is I, N, S, T or Y; X₆ is D, V, or Y; and X7 is A, F, P, S, V or W.
* In consensus sequence of CDR2: X₁ is I or V; X₂ is A, G, H, L, R, S, T, or V, X₃ is R, S, or W; X₄ is G, N, S, or Y; X₅ is A, G, L, or absent; X₆ is A, D, G, L, or S; X₇ is G, M, S, T, or V; and X₈ is A, L, or T.
* In consensus sequence of FR1: X₁ is E or Q; X₂ is Q or R; X₃ is G or D; X₄ is A, T, or V; and X₅ is A, G, or V.
* In consensus sequence of FR2: X₁ is L, M, R, or V; X₂ is A, G, or H; X₃ is F, L, or Y; X₄ is A or G; X₅ is E, N, Q, or R; X₆ is F or L; X₇ is A, G, or S; and X₈ is A, G or S.
* In consensus sequence of FR3: X₁ is L, N, or Y; X₂ is A, G, L, P, or Q; X₃ is M, V, or absent; X₄ is E or K; X₅ is G, K, or N; X₆ is A, G, T, or V; X₇ is E, K, or R; X₈ is D, N, or S; X₉ is H, Q or R; X₁₀ is D, N, or S; and X₁₁ is N, T or V
* In consensus sequence of FR4: X₁ is D, E, L, R, or T; X₂ is D, S, or Y; X₃ is G or A; and X₄ is L or Q.

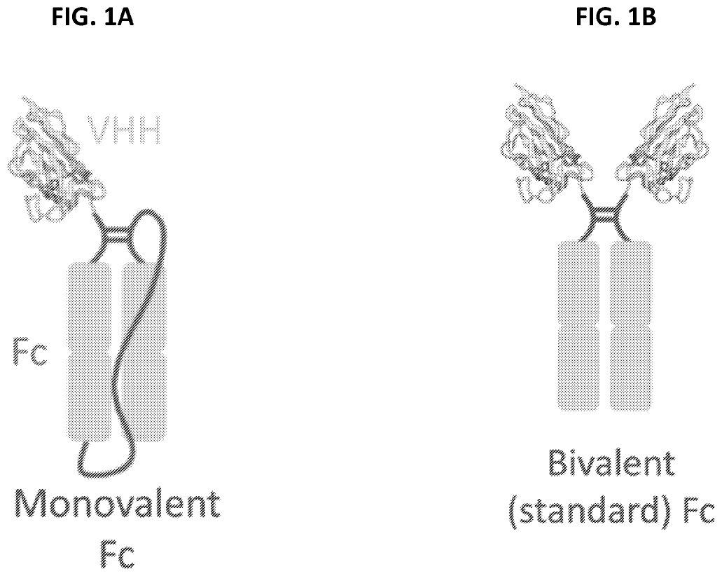

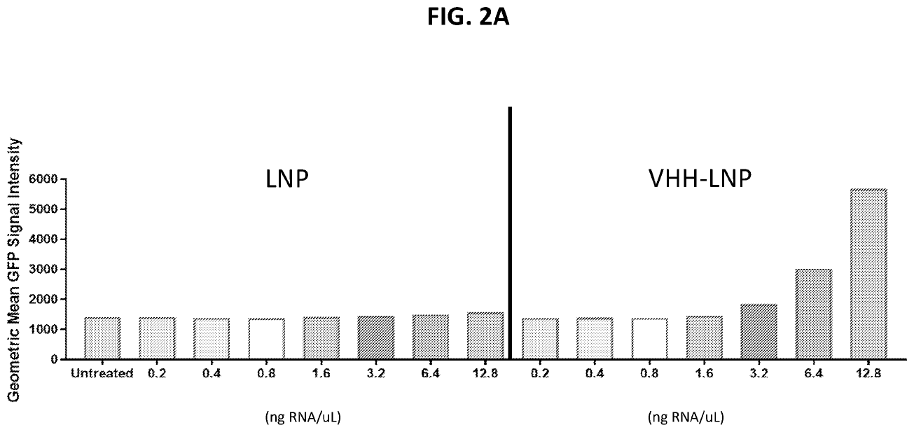

In some embodiments, the anti-CD117 antibody disclosed herein may be fused to an Fc fragment of an immunoglobulin molecule. Such Fc-fusion anti-CD117 antibodies may be in a monovalent format (see, e.g., FIG. 1A). Alternatively, the Fc-fusion anti-CD117 antibodies may be in a divalent format (see, e.g., FIG. 1B).

### B. Preparation of Anti-CD117 Antibodies

The anti-CD117 antibodies disclosed herein can be made by any method known in the art. See, for example, Harlow and Lane, (1998) Antibodies: A Laboratory Manual, Cold Spring Harbor Laboratory, New York. In some instances, high affinity anti-CD117 antibodies may be identified and characterized following conventional screening strategies. See also Example 1 below.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence, to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively-labeled antigen fragments is then determined by immunoprecipitation and gel electrophoresis. Certain epitopes can also be identified by using large libraries of random peptide sequences displayed on the surface of phage particles (phage libraries).

Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of CD117 have been replaced (swapped) with sequences from a closely related, but antigenically distinct protein (such as another member of the tumor necrosis factor receptor family). By assessing binding of the antibody to the mutant CD117, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

In some examples, an anti-CD117 antibody as disclosed herein can be prepared by recombinant technology as exemplified below.

Generally, a nucleic acid sequence encoding any of the anti-CD117 antibodies disclosed herein can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

In some instances, the coding sequence of the anti-CD117 antibody may be codon-optimized based on the expression system used for producing the antibody. Such codon-optimized coding sequences are also within the scope of the present disclosure.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40) (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10 (16): 1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColEl for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

A vector (e.g., an expression vector) comprising nucleic acids encoding any of the anti-CD117 antibodies may be introduced into suitable host cells for producing the antibodies. The host cells can be cultured under suitable conditions for expression of the antibody. Such antibodies, protein complexes, or polypeptide chains thereof can be recovered from the cultured cells (e.g., from the cells or the culture supernatant) via a conventional method, e.g., affinity purification. If necessary, polypeptide chains of the antibody or protein complex can be incubated under suitable conditions for a suitable period of time allowing for production of the antibody.

Standard molecular biology techniques are used to prepare the recombinant expression vector, transfect the host cells, select for transformants, culture the host cells and recovery of the antibodies from the culture medium. For example, some antibodies (in Fc-fusion format) can be isolated by affinity chromatography with a Protein A or Protein G coupled matrix.

Any of the nucleic acids encoding the anti-CD117 antibody as described herein, vectors (e.g., expression vectors) containing such; and host cells comprising the vectors are within the scope of the present disclosure.

### II. Cargo Delivery Vehicles

In some aspects, provided herein are cargo delivery vehicles comprising one or more of the anti-CD117 antibodies disclosed herein (e.g., one or more of the exemplary VHH anti-CD117 antibodies such as P10, P31, or P38) attached to a vehicle (e.g., an LNP), which can encapsulate or be associated with a cargo to be delivered to CD117⁺ cells such as hematopoietic cells. In some instances, the cargo can be one or more components of a gene editing system. The cargo delivery vehicles disclosed herein, carrying a gene editing system, is expected to efficiently deliver the gene editing system to CD117⁺ cells such as hematopoietic cells for genetic editing of a target gene, thereby treating a genetic disease associated with the target gene.

### A. Lipid Nanoparticles (LNPs)

As used herein, the term “lipid nanoparticle” or “LNP” refers to a particle comprising one or more lipids. In some embodiments, the lipid nanoparticle comprises a monolayer lipid membrane. Examples of such LNPs include micelle and reverse micelles. In other embodiments, the LNP comprises one or more bilayer lipid membranes. In some embodiments, the LNP disclosed herein is a liposome (also known as unilamellar liposome). Liposome refers to a spherical chamber or vesicle, which contains a single bilayer of an amphiphilic lipid or a mixture of such lipids surrounding an aqueous core. In other embodiments, the LNP is a multilamellar vesicle, which contains multiple lamellar phase lipid bilayers. Still in other embodiments, the LNP is solid lipid nanoparticle, which comprises a solid lipid core matrix that can solubilize lipophilic molecules. In some instances, a solid lipid nanoparticle can also be used to solubilize molecules such as nucleic acid, which may be encapsulated based on charges. In a solid lipid nanoparticle, the lipid core can be stabilized by surfactants (emulsifiers) and cargos can be distributed into lipid core.

Lipid nanoparticles include, but are not limited to, liposomes and micelles. Any of a number of lipids may be present, including cationic lipids, ionizable lipids, anionic lipids, neutral lipids, amphipathic lipids, conjugated lipids (e.g., PEGylated lipids), and/or structural lipids. Such lipids can be used alone or in combination.

In some embodiments, the lipid nanoparticle comprises a cationic lipid. As used herein, the term “cationic lipid” refers to any lipid that can be positively charged. Such cationic lipids can be ionizable or non-ionizable. In some embodiments, the lipid nanoparticles comprise an ionizable lipid, e.g., an ionizable cationic lipid, for example, DODMA. In some examples, the ionizable lipid is an ionizable amino lipid. The ionizable amino lipid may have at least one protonatable group. In some embodiments, the lipid nanoparticle comprises a non-ionizable lipid, e.g., a non-ionizable cationic lipid, for example, DOTAP. Ionizable lipid may be selected from, but not limited to, an ionizable lipid described in WO2013086354 and WO2013116126, the relevant disclosures of which are incorporated by reference for the subject matter and purpose referenced herein.

In some embodiments, the lipid nanoparticle comprises an anionic lipid. Anionic lipids suitable for use in lipid nanoparticles of the disclosure include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, phosphatidylserine, and other anionic modifying groups joined to neutral lipids.

In some embodiments, the lipid nanoparticle comprises one or more amphiphatic lipid, i.e., a lipid having a polar part and a non-polar part. Exemplary amphipathic lipids suitable for use in nanoparticles of the disclosure include, but are not limited to, sphingolipids, phospholipids, fatty acids, and amino lipids.

Particular amphipathic lipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition to pass through the membrane permitting.

In some examples, the lipid nanoparticle may comprise one or more amphiphatic lipids, which may be phospholipids, for example, one or more saturated or (poly) unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

In some embodiments, the lipid nanoparticle comprises PEGylated lipid. The lipid component of a lipid nanoparticle composition may include one or more molecules comprising polyethylene glycol, such as PEG or PEG-modified lipids. Such species may be alternately referred to as PEGylated lipids. A PEGylated lipid (also known as a PEG lipid or a PEG-modified lipid) is a lipid modified with polyethylene glycol. A PEGylated lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEGylated lipid may be PEG-c-DOMG, PEG-DMG, PEG-DLPE, PEG-DMPE, PEG-DPPC, PEG-DSG, or a PEG-DSPE lipid.

The lipid nanoparticle disclosed herein can comprise one or more structural lipids. As used herein, the term “structural lipid” refers to sterols and also to lipids containing sterol moieties. Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol.

### B. Methods of Preparing LNP-Antibody Conjugates

Standard methods for coupling the anti-CD117 antibody to LNPs may be used. For example, antibody-targeted LNPs can be constructed using, for instance, LNPs that incorporate a moiety to which the antibody binds (see, e.g., Renneisen et al., J. Bio. Chem., 265:16337-16342, 1990 and Leonetti et al., Proc. Natl. Acad. Sci. (USA), 87:2448-2451, 1990). Other examples of antibody conjugation are disclosed in U.S. Pat. No. 6,027,726, the relevant disclosures of which are incorporated by reference for the subject matter and purpose referenced herein. In another example, the anti-CD117 antibody can be attached to the LNPs via covalent bonds (see, for example Heath, Covalent Attachment of Proteins to Liposomes, 149 Methods in Enzymology 111-119 (Academic Press, Inc. 1987)). Other targeting methods include the biotin-avidin system.

In some aspects, provided herein is a method of conjugating the anti-CD117 antibody to LNPs using a sortase-mediated approach to functionalize the antibody, i.e., conjugating a functional group to the antibody, which can them form a covalent bond with a moiety on the LNP via a chemical reaction. See, e.g., Example 2 below.

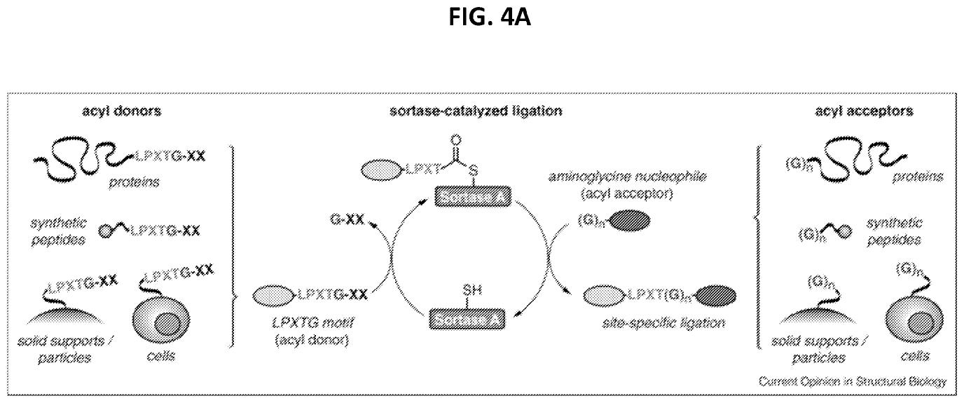

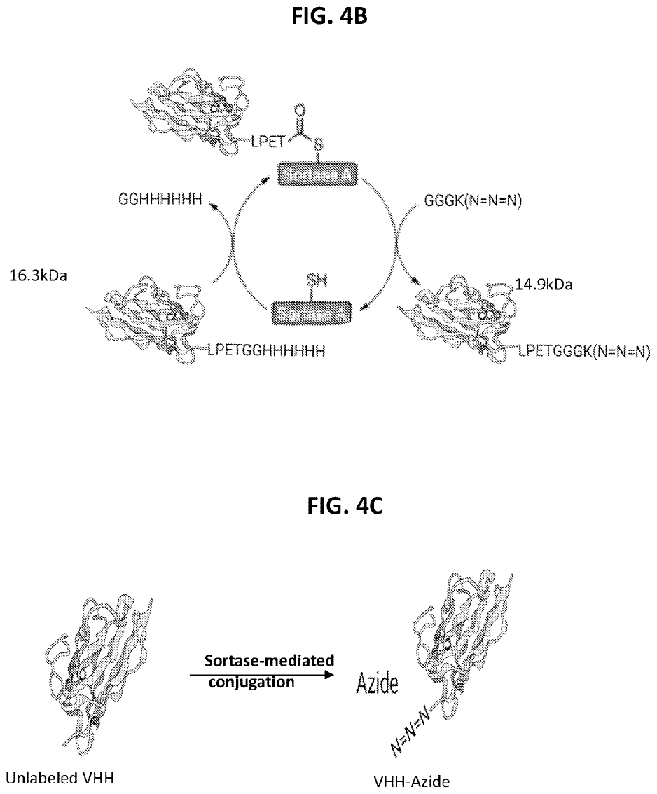

Sortase is a group of prokaryotic enzymes that modify surface proteins by recognizing and cleaving a carboxyl-terminal sorting signal. Sortases-mediated transacylation reactions, and their use in transacylation (sometimes also referred to as transpeptidation) for protein engineering are well known to those of skill in the art, see, e.g., WO2010087994 and WO2011133704, the relevant disclosures of each of which are incorporated by reference for the subject matter and purpose referenced herein. In general, the transpeptidation reaction catalyzed by sortase results in the ligation of species containing a transamidase recognition motif with those bearing one or more N-terminal glycine residues. For example, many sortases (e.g., Sortase A) recognize the motif LPXTG (SEQ ID NO: 88) (X being any amino acid residue) located at the C-terminus of a polypeptide. In one example, the sortase recognition motif may comprise LPETG (SEQ ID NO: 92) (e.g., LPETGG (SEQ ID NO: 89)). Sortase-mediated transacylation reactions are catalyzed by the transamidase activity of sortase. During the reaction, the sortase cleaves between the T and G residues in the recognition motif to generate the fragment of LPXT and the free T residue is then linked to a sortase peptide substrate, which typically contains a polyglycine fragment (e.g., a GGG fragment). Exemplary sortase-mediated processes for site specific modification of biomolecules, e.g., anti-CD117 VHH antibodies as disclosed herein, are provided in FIGS. 4A-4C.

To make a functionalized anti-CD117 antibody, any of the anti-CD117 antibody as disclosed herein can be modified to insert a sortase recognition motif (e.g., the LPXTG (SEQ ID NO: 88) motif) to the C-terminus of the antibody chain (e.g., via conventional recombinant technology). A sortase peptide substrate, which is modified to carry a functional group, can be used in the sortase reaction. The modified anti-CD117 antibody and the sortase peptide substrate can be incubated in the presence of a suitable sortase under conditions allowing for the transacylation reaction to occur, resulting in conjugation of the sortase peptide to the C-terminus of the antibody. Via the sortase-mediated reaction, the functional group contained in the sortase peptide substrate can be attached to the antibody to form a functionalized antibody.

A functional group refers to any chemical group capable of reacting with another group to form a covalent bond. Examples include, but are not limited to, a thiol group, an amine group, an azide group, a thiol group, or a DBCO group. In some instances, the functional group can also be a member of a receptor-ligand pair, for example, biotin or streptavidin. In one example, the sortase peptide substrate comprises a motif of GGGK (SEQ ID NO: 93) (N3), in which the K residue is modified by the N3 functional group to make it an azide functionalized substrate. Via the sortase-mediated reaction, the azide functional group can be conjugated to the anti-CD117 antibody.

In some instances, a functionalized anti-CD117 antibody as disclosed herein can be incubated with an LNP carrying a functional group (first functional group) that can react with its complementary functional group (second functional group) contained in the functionalized anti-CD117 to form a covalent bond. As used herein, “a functional group” and its “complementary functional group” refer to a pair of functional groups that can react to form a covalent bond. In some instances, the LNP may comprise PEG-lipid molecules, which are functionalized to carry the first function group. In some instances, the anti-CD117 antibody may have the second functional group attached to one or more internal amino acid residues (e.g., internal lysine residues). Alternatively or in addition, the anti-CD117 antibody may have the second functional group attached to its C-terminal residue (e.g., via a sortase reaction as disclosed herein).

In some examples, a functionalized anti-CD117 antibody may carry an azide functional group and the LNP may carry PEG-conjugated lipid molecules, in which the PEG moiety is functionalized with polyethylene glycol (DBCO-PEG), so the azide group can form a covalent bond with the DBCO functional group attached to the PEG moiety. In other examples, a functionalized anti-CD117 antibody may carry one or more thiol groups attached to one or more internal amino acid residues and/or terminal residues in the antibody (e.g., attached to one or more lysine residues). The LNP may carry a maleimide functional group (e.g., attached to PEG-lipid molecules contained in the LNP). Upon reaction between the thiol group and the maleimide group to form covalent bonds, the anti-CD117 antibody can be conjugated to the LNP.

The PEG moiety may be of a suitable size. In some instances, PEG2000 can be used. Alternatively, the functionalized anti-CD117 antibody may be incubated with a PEG-conjugated lipid molecule (see, e.g., Example 2 below) to allow for covalent conjugation of the antibody to the PEG-lipid molecule. Such a conjugate can then be incubated with LNPs to allow for incorporation of the antibody/PEG-lipid conjugate into the LNPs.

Alternatively, an anti-CD117 antibody may be functionalized to conjugate to a PEG-lipid (e.g., PEG-DBCO) and the LNPs may be functionalized to carry a functional group such as an azide group. Covalent bonds can be formed between the VHH-DBCO-PEG-Lipid and the LNP-azide to conjugate the VHH to the LNPs.

Other methods known in the art can also be used to produce antibody-LNP conjugates as disclosed herein. For example, the conjugation may be mediated by a ligand-receptor pair, e.g., a biotin-streptavidin pair. In this case, one member of the ligand-receptor pair may be linked to the anti-CD117 VHH antibody and the other member may be linked to the LNPs, directly or indirectly. Via binding between the ligand and receptor, the anti-CD117 antibody can be conjugated to the LNPs.

### C. Exemplary Cargos for Delivery

The LNPs disclosed herein may carry a cargo to be delivered to target cells. In some instances, the cargo can be a therapeutic agent (e.g., peptide, polypeptide, protein, nucleic acid, small molecule, etc.) or can produce a therapeutic agent, for example, an expression cassette designed for expressing the therapeutic agent. Alternatively, the cargo may be a diagnostic agent. In specific examples, the cargo may comprise one or more components of a gene editing system. As used herein, a gene editing system refers to compositions comprising protein component(s) and optionally nucleic acid component(s) required for conducting genetic editing at a target genetic site as designed. Any of the cargo disclosed herein can be loaded into the LNPs (e.g., encapsulated by or attached to) by conventional methods.

### Gene Editing System

In some embodiments, the cargo carried by any of the delivery vehicles disclosed herein may be a gene editing system. Any suitable gene editing system known in the art can be used in the delivery system disclosed herein, for example, the nuclease-dependent targeted editing using zinc-finger nucleases (ZFNs) system, the transcription activator-like effector nucleases (TALENs) system, or the RNA-guided CRISPR-Cas9 nucleases system (CRISPR/Cas9; Clustered Regular Interspaced Short Palindromic Repeats Associated 9). In specific examples, the gene editing system is a CRISPR-Cas9-mediated gene editing system.

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (crRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM).

TracrRNA hybridizes with the 3′ end of crRNA to form an RNA-duplex structure that is bound by the Cas9 endonuclease to form the catalytically active CRISPR-Cas9 complex, which can then cleave the target DNA. Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end). After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

### (a) Cas9

In some embodiments, the gene editing system carried by the delivery vehicle disclosed herein comprises a Cas9 (CRISPR associated protein 9) endonuclease or a nucleic acid encoding the Cas9 enzyme. Cas9 can be used in a CRISPR method for making the genetically engineered T cells as disclosed herein. The Cas9 enzyme may be one from Streptococcus pyogenes, although other Cas9 homologs may also be used. It should be understood, that wild-type Cas9 may be used or modified versions of Cas9 may be used (e.g., evolved versions of Cas9, or Cas9 orthologues or variants), as provided herein. In some embodiments, Cas9 comprises a Streptococcus pyogenes-derived Cas9 nuclease protein that has been engineered to include C- and N-terminal SV40 large T antigen nuclear localization sequences (NLS). The resulting Cas9 nuclease (SNLS-spCas9-sNLS) is a 162 kDa protein that is produced by recombinant E. coli fermentation and purified by chromatography. The spCas9 amino acid sequence can be found as UniProt Accession No. Q99ZW2, which is incorporated by reference for the subject matter and purpose referenced herein.

(b) Guide RNAs (gRNAs)

In some instances, the gene editing system disclosed herein further includes a nucleic acid component, such as an RNA molecule or a nucleic acid encoding such, which guides the endonuclease to cleave at the genetic target site as designed. For example, A CRISPR-Cas9-mediated gene editing system as described herein includes a guide RNA or a gRNA. As used herein, a “gRNA” refers to a genome-targeting nucleic acid that can direct the Cas9 to a specific target sequence within the genetic target site for gene editing at the specific target sequence. A guide RNA comprises at least a spacer sequence that hybridizes to a target nucleic acid sequence within a target gene for editing, and a CRISPR repeat sequence.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011). In some embodiments, the genome-targeting nucleic acid (e.g., gRNA) is a double-molecule guide RNA. In some embodiments, the genome-targeting nucleic acid (e.g., gRNA) is a single-molecule guide RNA.

A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

The “target sequence” is in a target gene that is adjacent to a PAM sequence and is the sequence to be modified by Cas9. The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence. The spacer of a gRNA interacts with a target nucleic acid of interest in a sequence-specific manner via base pairing. The nucleotide sequence of the spacer thus varies depending on the target sequence of the target nucleic acid of interest.

In a CRISPR/Cas system herein, the spacer sequence is designed to base-pair with a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNRG-3′, the target nucleic acid can be the sequence that corresponds to the Ns, wherein N can be any nucleotide, and the underlined NRG sequence is the S. pyogenes PAM.

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to 1 mismatch.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be a sgRNA, which may comprise a 20-nucleotide spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence.

In some embodiments, the sgRNA comprises no uracil at the 3′ end of the sgRNA sequence. In other embodiments, the sgRNA may comprise one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as a sgRNA can comprise one or more 2′-O-methyl phosphorothioate nucleotides, which may be located at either the 5′ end, the 3′ end, or both.

In certain embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

It should be understood that more than one suitable Cas9 and more than one suitable gRNA can be used in methods described herein, for example, those known in the art or disclosed herein. In some embodiments, methods comprise a Cas9 enzyme and/or a gRNA known in the art. Examples can be found in, e.g., WO 2019/097305A2, and WO2019/215500, the relevant disclosures of each of which are herein incorporated by reference for the purposes and subject matter referenced herein.

### (c) Donor Template

In some embodiments, the gene editing system to be carried by the delivery vehicle may further comprise a donor template, which may be located on a viral vector such as an adeno-associated viral (AAV) vector. AAVs are small viruses which integrate site-specifically into the host genome and can therefore deliver a transgene, such as CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, the AAV for use in delivering the CAR-coding nucleic acid is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

The donor template may comprise a gene of interest (GOI). In some instances, the GOI is to be knocked-in at the genetic site where the gene editing is designed. Such a GOI may encode a therapeutic protein for expressing in the target cell. In some instances, the therapeutic protein compensates the loss of protein activity due to a genetic defect in the target cell. In some examples, the donor template may carry a GOI, which is a functional counterpart of the defective gene in the target cell. In other examples, the donor template may comprise a GOI, which comprises a nucleotide sequence to replace a region comprising the genetic defect to be fixed by the designed gene editing.

A donor template as disclosed herein contain an upstream arm and/or a downstream art flanking the GOI. The upstream and downstream arms share sufficient homologies to a genomic target site to allow for efficient homology-directed repair (HDR) using the CRISPR-Cas9 gene editing technology, thereby incorporating the GOI into the genomic target site. In this case, both strands of the DNA at the target locus can be cut by a CRISPR Cas9 enzyme guided by gRNAs specific to the target locus. HDR then occurs to repair the double-strand break (DSB) and insert the donor DNA carrying the GOI. For this to occur correctly, the upstream and/or downstream arms can be designed with flanking residues which are complementary to the sequence surrounding the DSB site in the target gene (homology arms). These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template, in some embodiments, can be inserted at a site nearby an endogenous promoter (e.g., downstream or upstream) so that its expression can be driven by the endogenous promoter. In other embodiments, the donor template may comprise an exogenous promoter and/or enhancer, for example, a constitutive promoter, an inducible promoter, or tissue-specific promoter to control the expression of the GOI. In some embodiments, the exogenous promoter is an EFla promoter. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

### III. Methods for Cargo Delivery

Any of the CD117-targeting delivery vehicles disclosed herein, e.g., anti-CD117 VHH antibody-conjugated LNPs, can be used to deliver the cargo carried by the vehicle (e.g., a gene editing system) to CD117⁺ cells such as hematopoietic stem cells. Such delivery vehicles can be formulated to form pharmaceutical compositions, which can be administered to a subject in need of the treatment or diagnosis by the cargo via a suitable administration route.

Any of the CD117-targeting delivery vehicles disclosed herein, e.g., anti-CD117 VHH antibody-conjugated LNPs, may be mixed with one or more pharmaceutically acceptable carrier, adjuvant, or excipient to form a pharmaceutical composition, which is also within the scope of the present disclosure. The amount of the delivery vehicle can be in an amount effective for delivering the cargo carried thereby, which can be used to achieve the intended therapeutic or diagnostic purposes (e.g., editing a genetic target site and/or treating a relevant disease, disorder, or condition in a patient in need thereof).

The term “pharmaceutically acceptable carrier, adjuvant, or vehicle” refers to a non-toxic carrier, adjuvant, or vehicle that does not destroy the pharmacological activity of the therapeutic-loaded hydrogel with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition as disclosed herein may be administered via a suitable route, for example, by intravenous administration or by local injection. Alternatively, the composition may be administered via a parenteral route. The term “parenteral” as used herein includes subcutaneous, intravenous, intramuscular, intra-articular, intra-synovial, intrasternal, intrathecal, intrahepatic, intralesional and intracranial injection or infusion techniques.

In some embodiments, the pharmaceutical compositions can be administered by an intravenous, subcutaneous, intranasal, inhalation, intramuscular, intraocular, intraperitoneal, intratracheal, transdermal, buccal, sublingual, rectal, topical, local injection, or surgical implantation route.

To aid in delivery of the pharmaceutical composition disclosed herein, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions.

Other commonly used surfactants, such as polysorbates (Tween® compounds), sorbitan esters (Span® compounds) and other emulsifying agents or bioavailability enhancers which are commonly used in the manufacture of pharmaceutically acceptable solid, liquid, or other dosage forms may also be used for the purposes of formulation.

Alternatively, the pharmaceutical compositions disclosed herein may be administered in the form of suppositories for rectal administration. These can be prepared by mixing the delivery vehicle with a suitable non-irritating excipient that is solid at room temperature but liquid at rectal temperature and therefore will melt in the rectum to release the drug. Such materials include cocoa butter, beeswax and polyethylene glycols.

The pharmaceutical compositions disclosed herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

It should also be understood that a specific dosage and treatment regimen for any particular patient will depend upon a variety of factors, including the activity of the specific therapeutic that is present in the modified hydrogel, the age, body weight, general health, sex, diet, time of administration, rate of excretion, drug combination, and the judgment of the treating physician and the severity of the particular disease being treated. The amount of a therapeutic in the hydrogels of the present disclosure in the composition may also be dependent upon the particular therapeutic in the composition.

Any of the pharmaceutical composition disclosed herein may be used to deliver the cargo contained in the delivery vesicle (e.g., LNPs) to a subject to allow for delivery of the cargo into host cells and produce therein the encoded agents of interest, such as therapeutic nucleic acids or therapeutic proteins, or exert the intended therapeutic or diagnostic purposes (e.g., gene editing).

To practice the methods described herein, an effective amount of pharmaceutical compositions, comprising any of the cargo-loaded, CD117-targeting delivery vesicles disclosed herein, can be administered to a subject in need of the treatment via a suitable route, e.g., those described herein. The delivery vehicles would be effective in achieving the intended therapeutic or diagnostic purposes, for example, for editing a target gene.

The term “patient” or “subject” as used herein, means an animal, for example a mammal, such as a human. In some instances, the patient is a human patient, who may have a disease that can be treated by a cargo contained in a CD117-targeting delivery vehicle as disclosed herein. In some instances, the cargo is a gene editing system designed for genetic edit of a target gene. The human patient may have a disease associated with the target gene. The delivery vehicle disclosed herein may be used for gene therapy in CD117⁺ cells such as hematopoietic stem cells.

### IV. Kits for Use in Cargo Delivery

The present disclosure also provides kits for use of any of the CD117-targeting delivery vehicles to deliver a cargo of interest (e.g., a gene editing system) to CD117⁺ cells such as HSCs to achieve intended therapeutic or diagnostic purposes. Such kits may include one or more containers comprising one or more pharmaceutical compositions that comprise one or more of the CD117-targeting delivery vehicles disclosed herein, and one or more pharmaceutically acceptable carriers. In some examples, the CD117-targeting delivery vehicle carries a gene editing system such as a CRISPR/Cas9-mediated gene editing system as those disclosed herein.

In some embodiments, the kit can comprise instructions for use in any of the methods described herein. The included instructions can comprise a description of administration of the pharmaceutical compositions to a subject to achieve the intended activity in a human patient. The kit may further comprise a description of selecting a human patient suitable for treatment based on identifying whether the human patient is in need of the treatment. In some embodiments, the instructions comprise a description of administering the pharmaceutical compositions to a human patient who is in need of the treatment.

The instructions relating to the use of any of the delivery vehicles for delivering the cargo contained therein and for achieving the intended therapeutic or diagnostic purposes include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the delivery vehicles are used for delivering a cargo of interest for treating, delaying the onset, and/or alleviating a target disease in a subject. When the cargo is a gene editing system, the label or package insert indicates that the delivery vehicle is for use in editing a target genetic site in CD117⁺ cells such as HSCs by the gene editing system carried thereby.

The kits provided herein are in suitable packaging. Suitable packaging includes, but is not limited to, vials, bottles, jars, flexible packaging, and the like. Also contemplated are packages for use in combination with a specific device, such as an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a population of the genetically engineered T cells as disclosed herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A Practical Approach, Volumes I and II (D. N. Glover ed. 1985); Nucleic Acid Hybridization (B. D. Hames & S. J. Higgins eds. (1985>>; Transcription and Translation (B. D. Hames & S. J. Higgins, eds. (1984»; Animal Cell Culture (R. I. Freshney, ed. (1986>>; Immobilized Cells and Enzymes (IRL Press, (1986»; and B. Perbal, A Practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Screening for and Characterization of Anti-CD117 VHH Antibodies

This example discloses screening for and characterization of anti-CD117 VHH antibodies by assays such as ELISA, Octet®, and cell binding assays.

### Methods

### (i) Preparation of Antibodies

Nucleotide sequences encoding a total of 28 anti-CD117 VHH antibodies were each cloned into a vector for expression and secretion of the encoded VHH antibody in Expi293 cells according to manufacturer's protocol (ThermoFischer Scientific). The host cells were transfected with the expression vector and the transfected cells were maintained in a shaking incubator for about one week to allow expression and secretion of the VHH antibody into the supernatant. The cell supernatants were harvested by centrifugation and sterile-filtered (0.2 μM) to remove cell debris. Standard protein analytics were performed to estimate supernatant protein expression and yield.

Human codon-optimized coding sequences for certain VHH antibodies were used in producing the VHH antibodies. Examples are provided below:

Codon-Optimized Coding Sequence for P10 (signal
sequence not included)
(SEQ ID NO: 95)
CAAGTGCAGCTGGTGGAATCCGGAGGAGGACTGGTGCAAGCCGGCGGATC
TCTGAGACTGAGCTGTGTGGCCAGCGGAAGAACCTTCAGCAGCTACGCCA
TGGGCTGGTTTAGACAAGGCCCCGGCAAAGAGAGGGAGTTTGCCGCCGCC
ATTCTGTCCAACGGACTGACAACCTACTACGCCGACAGCGTGAAGGGAAG
ATTCACCATCAGCAGAGACAACGCCAAGGACACCGTGTATCTGCAGATGA
ACTCTCTGAAGCCCGAGGACACCGCCAACTACTACTGCGCCGCCGCCTTT
ACCCCCGAGTTTAGGGACGGCGGCATCTGGGACGATGCCAGCGTGGACTA
CTGGGGCCAAGGCACACAAGTGACCGTGTCCAGC
Codon-Optimized Coding Sequence for P38 (signal
sequence not included)
(SEQ ID NO: 96)
GAGGTGCAGCTGGTGGAGAGCGGAGGAGGACTGGTGCAAGCCGGAGGCTC
TCTGAGACTGAGCTGTGCCGCCAGCGGAAGAACCAGCGGCAGCTACGTGA
TGGGCTGGTTCAGACAAGCCCCCGGCAAGGAGAGGGAATTCGTGGCCGCC
ATTAGCTGGAGCGCCGGCATGACCTACTACGCCGACAGCGTGAAGGGAAG
ATTCACCATCTCTAGAGACAACGCCAAGAACACCGTGTATCTGCAGATGA
ACTCTCTGAAGCCCGAGGACACCGCCGTGTACTACTGTGCCGCCGATCAG
AGAGGCGTGCCCGCCTACTACTCCGACTACGCTCTGTACGACTACTGGGG
CCAAGGCACCCAAGTGACCGTGAGCAGC
Codon-Optimized Coding Sequence for P32 (signal
sequence not included)
(SEQ ID NO: 97)
CAAGTGCAGCTGGTGGAAAGCGGCGGAGGACTGGTGCAAGCCGGAGACTC
TCTGAGACTGAGCTGCGCCGCCAGCGGCAGAACCTTCAGCAGCTACGCCA
TGGGCTGGTTCAGACAAGCCCCCGGCAAGGAGAGAGAGTTCGTGGCCAGC
ATCACCAGCAGCGGACTGGTGGCCTACTACGGAGACTCCGTGGAGGCCAG
ATTCACCATCTCTAGAGACAACGCCAAGAATACCGTGTATCTGCAGATGG
ACTCTCTGAAGCCCGAGGACACCGCTGTGTACTACTGTGCTGCTAGACAG
TATGTGGGAAGCGGCTCCTACTATCTGAAGAAGGAGGGCGGCTACGAGTA
CTGGGGACCCGGCACCCAAGTGACCGTGTCCAGC

Nine VHH antibodies, P10, P12, P27, P29, P31, P32, P35, P37, or P38, identified herein as having high binding activity to human CD117, and optionally cynomolgus CD117, were expressed as fusion polypeptides with an Fc fragment. The amino acid sequences of these VHH antibodies are provided in Table 1 above. See also the sequence alignment below. These Fc-fusion polypeptides were either in monovalent format as depicted in FIG. 1A or in bivalent format as depicted in FIG. 1B. The following table summarizes the monovalent antibody/bivalent antibodies to the VHH antibodies.

TABLE 2
VHH Antibodies and Correspondence Monovalent
and Bivalent Fc-Fusion Antibodies

| VHH | Monovalent | Bivalent |
|---|---|---|
| P10 | P-0010 | P-0053 |
| P12 | P-0012 | P-0059 |
| P27 | P-0027 | P-0060 |
| P29 | P-0029 | P-0054 |
| P31 | P-0031 | P-0055 |
| P32 | P-0032 | P-0057 |
| P35 | P-0035 | P-0056 |
| P37 | P-0037 | P-0058 |
| P38 | P-0038 | P-0072 |

The monovalent and bivalent VHH-Fc fusion antibodies were purified for kinetic analysis, cell binding, and other characterization assays described below.

### (ii) Preparation of CD117 Ectodomains

Human and cynomolgus monkey (cyno) CD117 ectodomain was expressed by the same approach as disclosed herein. The CD117 ectodomain was expressed as a fusion polypeptide to an AviTag or a His tag added to the C terminus to allow capture on streptavidin biosensors.

In some instances, biotin was added to a cell culture of Expi293F cells at the same time a DNA plasmid was transfected into the cells. The DNA plasmid carries coding sequences of a target protein (e.g., the CD117 ectodomain) and a biotin ligase BirA. This expression system produces biotinylated target protein, such as biotinylated CD117 ectodomain.

(iii) Biolayer Interferometry Octet® Assay

Pre-equilibrated streptavidin biosensors (SA, from Sartorius) were coated with biotinylated CD117 ectodomain by dipping the biosensors into wells containing an optimized dilution of human or cyno CD117 ectodomain. After re-equilibrating the biosensor tips in assay buffer, the biosensors were dipped into supernatant containing the VHH antibodies to allow association between the antibodies and the CD117 ectodomain. Finally, the biosensors were dipped into the assay buffer. The VHH antibodies disassociated from the CD117 ectodomain if association occurred. The association and dissociation of each VHH antibody was characterized qualitatively to identify VHHs that could bind both human and cyno CD117 ectodomains.

### (iv) ELISA and Flow Cytometry Analysis

The ELISA and flow cytometry assays were performed following route practice.

### (v) SCF Competition Assay

Full length AviTagged CD117 ectodomain was bound to a streptavidin (SA) biosensor (Sartorius). The biosensor was either (1) incubated first with SCF (R&D systems), followed by one of the 9 top VHH antibodies disclosed herein, (2) incubated first with one of the VHH antibodies, followed by SCF.

### (vi) Competition Assay Between VHH Antibodies

Full length AviTagged CD117 was bound to SA biosensor. The biosensor was then incubated with one VHH, allowed to saturate, rinsed, followed by incubation with a second VHH. If the second VHH was able to bind (no competition), the two VHHs were considered to bind different locations, and thus be in different epitope bins. If the second VHH is unable to bind, the second VHH was considered to be in the same bin as the first VHH.

The saturation step was performed in Octet assays. In each assay, the saturating amount of the loading material (e.g., the CD117 ectodomain supernatant or the purified anti-CD117 VHH antibody) was optimized so as to maximize the amount of the loading material on the biosensor tips without underloading or overloading, both of which could negatively affect the results of association and dissociation observed in the assay.

### Results

The antibody-containing supernatants were screened concurrently for their ability to bind either the human or cyno CD117 ectodomain using Biolayer Interferometry (Octet®), and for their ability to bind CD117 expressing SKMEL 3 cells using flow cytometry. Binding activities of the VHH antibodies in both monovalent format (see FIG. 1A) and bivalent format (see FIG. 1B) were examined using the assays disclosed herein.

### (i) Identification of Top VHH Antibodies

Upon analyzing binding activities of the monovalent and bivalent VHH-Fc antibodies to CD117⁺ cells (SKMEL-3/CD34⁺ cells), cross-reactivity between human and cyno CD117, and other features examined, P10, P12, P27, P29, P31, P32, P35, P37, and P38 were identified as the top binders. A sequence alignment of these top nine top binders and a few additional VHH antibodies is provided below (CDRs in boldface).

```
                                                                        CDR1 CDR2
MP16 EVQLVESGGGLVQAGGSLRLSCVASGT-TSGIVAMHWYRQAPGKRRELVASVVRG-ASLN (SEQ
ID NO: 98) 58
P27/P60 EVQLVESGGGLVRAGGSLRLSCAASGRTTFSTYWLGWFRQAPGKNREFVAAISWSAGMAY (SEQ
ID NO: 99) 60
P35/P57 QVQLVESGGGLVQAGGSLRLSCAASGG-TFSIYPVGWFRQAPGKQREFVAAIGWSASGTY
(SEQ ID NO: 100) 59
P37/P58 QVQLVESGGGLVQAGDSLRLSCAVSGR-TLSNYFMAWLRQAPGKEREFVAAIHWSLGSTY
(SEQ ID NO: 101) 59
P32/P56 QVQLVESGGGLVQAGDSLRLSCAASGR-TFSSYAMGWFRQAPGKEREFVASITSSGLVAY
(SEQ ID NO: 102) 59
P29/P54 QVHLVESGGGLVQAGGSLGLSCAASGH-TFSNYAMGWFRQAPGKEREFVAAISWSGGSTL
(SEQ ID NO: 103) 59
P10/P53 QVQLVESGGGLVQAGGSLRLSCVASGR-TFSSYAMGWFRQGPGKEREFAAAILSNGLTTY (SEQ
ID NO: 104) 59
MP14 QVQLVESGGGLVQAGGSLRLSCAGSGR-TVSTYAMGWFRQAPGKEREFVSAIARSGGGTY
(SEQ ID NO: 105) 59
MP6 QVQLVESGGGLVQAGGSLRLSCVGSGR-TVSTYAMGWFRQAPGKERELVAAIGRSGGSTY
(SEQ ID NO: 106) 59
P12/P59 QVQLVESGGGLVQAGGSLRLSCTASGD-TFSSYSMGWFRQGPGKEREFVGGIRWSGGTTY
(SEQ ID NO: 107) 59
MP7 QVQLVESGGGLVQAGGSLRLSCAASGR-TFSSYVMGWFRQAPGKEREFVAAISWYGDSTY (SEQ
ID NO: 108) 59
P38/P72 EVQLVESGGGLVQAGGSLRLSCAASGR-TSGSYVMGWFRQAPGKEREFVAAISWSAGMY
(SEQ ID NO: 109) 59
P31/P55 EVQLVESGGGLVRAGGSLRLSCAASGR-TFTYDARAWFRQAPGKEREFVAAISWSGGSTY
(SEQ ID NO: 110) 59
                                                                                                            CDR3
MP16 YLDSVKGRFIISEDGARNTVYLQMNSLKPEDTAVYYCNAVLR------------TGMDYW (SEQ
ID NO: 111) 106
P27/P60 YQDS-KGRFTISRDNTKNTAYLQMNSLQPEDTAVYYCAAGRFHPI-------RVDTALYW (SEQ
ID NO: 112) 112
P35/P57 YGDSVEGRFTVSRDNARSTVYLRMSSLKPDDTAVYYCAAASGSN----WRLGAIDEYDSW (SEQ
ID NO: 113) 115
P37/P58 YQDPVKGRFTISRDKAKNTVYLQMNTLKPEDTATYICAAGQHLSGLGGSAWS-IEGDYW (SEQ
ID NO: 114) 117
P32/P56 YGDSVEARFTISRDNAKNTVYLQMDSLKPEDTAVYYCAARQYVG-SGSYYLKKEGGYEYW (SEQ
ID NO: 115) 118
P29/P54 YADSVKGRFTISRDNGENTVYLQMNSLKPEDTAVYYCALDSTGVYGTGYVSSRKGRYEYW (SEQ
ID NO: 116) 119
P10/P53 YADSVKGRFTISRDNAKDTVYLQMNSLKPEDTANYYCAAAFTPEFRDGGIW-DDASVDYW (SEQ
ID NO: 117) 118
MP14 YADSVKGRFTISRDNAKNTVYLQMNSLKPEDTAVYYCAASDS---Y--FYA-SPHLYDYW (SEQ
ID NO: 118) 113
MP6 YADSVKGRFTISRDNVRNTVYLQMNSLKPEDAAVYYCAASDT---Y--FYA-SPHLYTDW (SEQ
ID NO: 119) 113
P12/P59 YADSVKGRFTISRDNAKNTVYLQMNSLKREDTAVYYCAAVRRRWLI--W---QEEEYDYW (SEQ
ID NO: 120) 114
MP7 YADSVKGRFTISRDNAKNTVYLHMNSLKPEDTAVYYCAARRGTILV--V---QEYEYDYW (SEQ
ID NO: 121) 114
P38/P72 YADSVKGRFTISRDNAKNTVYLQMNSLKPEDTAVYYCAADQRGVPA--YYS-DYALYDYW (SEQ
ID NO: 122) 116
P31/P55 YPNSMKGRFTISRDNAKNTVYLQMNSLKPEDTAVYYCAADE-SFPA--YYS-DYALYRYW (SEQ
ID NO: 123) 115
```

| | | | |
|---|---|---|---|
| MP 16 | GQGTLVTVSS | (SEQ ID NO: 124) | 116 |
| P27/P60 | AQGTQVTVSS | (SEQ ID NO: 125) | 122 |
| P35/P57 | GQGTQVTVSS | (SEQ ID NO: 126) | 125 |
| P37/P58 | GQGTQVTVSS | (SEQ ID NO: 126) | 127 |
| P32/P56 | GPGTQVTVSS | (SEQ ID NO: 127) | 128 |
| P29/P54 | GQGTLVTVSS | (SEQ ID NO: 124) | 129 |
| P10/P53 | GQGTQVTVSS | (SEQ ID NO: 126) | 128 |
| MP14 | GQGTLVTVSS | (SEQ ID NO: 124) | 123 |
| MP6 | GQGTQVTVSS | (SEQ ID NO: 126) | 123 |
| P12/P59 | GQGTLVTVSS | (SEQ ID NO: 124) | 124 |
| MP7 | GQGTLVTVSS | (SEQ ID NO: 124) | 124 |
| P38/P72 | GQGTQVTVSS | (SEQ ID NO: 126) | 126 |
| P31/P55 | GQGTLVTVSS | (SEQ ID NO: 124) | 125 |

The binding affinities of the top nine antibodies identified herein to human CD117 (in both monovalent and bivalent forms) as determined by the ELISA assay are provided in Table 3 below.

TABLE 3
Binding Affinities of Top VHH Antibodies

| | Monovalent K_{D} (nM) | | Bivalent K_{D} (nM) |
|---|---|---|---|
| P-0010 | 0.28 | P-0053 | 0.16 |
| P-0012 | 1.04 | P-0059 | 0.06 |
| P-0027 | 1.32 | P-0060 | 0.14 |
| P-0029 | 2.48 | P-0054 | 0.54 |
| P-0031 | 0.52 | P-0055 | 0.22 |
| P-0032 | 1.49 | P-0057 | 0.21 |
| P-0035 | 1.91 | P-0056 | 0.36 |
| P-0037 | 0.97 | P-0058 | 0.33 |
| P-0038 | 1.00 | P-0072 | 0.21 |
| | | cKAb-1 | 0.12 |

Table 4 below shows the ECso values for the top 9 VHH antibodies to SKMEL cells and CD34⁺ cells (both CD117⁺ cells) as determined by flow cytometry.

TABLE 4
EC₅₀ Values of VHH Antibodies to CD117 on Cell Surfaces

| | Monomeric FC, | Monomeric FC, |
|---|---|---|
| | skMEL-3 cells | CD34⁺ HSC |
| | EC50 (nM) | EC50 (nM) |
| VHH | Human | Human |
| Monomeric FC | | |
| P-0010 | 2 | 2 |
| P-0012 | >10 | >10 |
| P-0027 | 10 | 10 |
| P-0029 | >50 | 50 |
| P-0031 | 3 | 4 |
| P-0032 | Low & no saturation | Low & no saturation |
| P-0035 | 10 | 30 |
| P-0037 | 20 | >50 |
| P-0038 | 10 | 10 |

| | Bivalent FC, | Bivalent FC, |
|---|---|---|
| | skMEL-3 | CD34⁺ HSC |
| | EC50 (nM) | EC50 (nM) |
| | Human | Human |
| Bivalent FC | | |
| P-0053 | 1 | 1 |
| P-0059 | 50 | 50 |
| P-0060 | 2 | 2 |
| P-0054 | >50 | >50 |
| P-0055 | 1 | 1 |
| P-0057 | Low & no saturation | Low & no saturation |
| P-0056 | 3 | 10 |
| P-0058 | 3 | 50 |
| P-0072 | 1 | 2 |

### (ii) Human-Cyno CD117 Cross-Reactivity

Results from flow cytometry and Octet® analysis show that the monovalent form of P10, P29, P31, P35, P37, and P38 cross-react between human and cyno CD117. The bivalent form of all of the top nine VHH antibodies cross-react between human and cyno CD117.

For example, kinetics parameters of the top VHH antibodies for binding to human CD117 and cynomolgus monkey CD117 ectodomain were obtained from the Octet® analysis by Biolayer Interferometry as described above. A minimum of three concentrations of purified VHH antibodies were tested per ectodomain target. The Octet® software was used to perform a global kinetic fit across all concentrations using a 1:1 binding model. This allowed determination of the kinetic parameters, association rate (kₒₙ), dissociation rate (k_{off}) and equilibrium constant (K_{D}) for the 9 antibodies of interest. Total chi-squared (X²) was also examined to determine the goodness of fit for the software's kinetic model.

The binding affinities of each of the VHH antibodies, in either the monovalent form or the bivalent form, are provided in Table 5 below.

TABLE 5
Binding Activity to Human and Cyno CD117

Monomeric FC K_{D} (nM) from Octet

| VHH | Human | Cyno |
|---|---|---|
| Monomeric FC | | |
| P-0010 | 16.5 | 54.9 |
| P-0012 | 49.2 | N/A |
| P-0027 | 44.7 | 8.11 |
| P-0029 | 37.4 | N/A |
| P-0031 | 7.42 | 1.62 |
| P-0032 | 1.86 | 8.01 |
| P-0035 | 2.89 | 7.02 |
| P-0037 | 28.02 | 25 |
| P-0038 | 8.85 | 18.97 |

Bivalent FC K_{D} (nM) from Octet

| | Human | Cyno |
|---|---|---|
| Bivalent FC | | |
| P-0053 | <0.001 | 1.36E−03 |
| P-0059 | 4.46E−02 | 1.12E−02 |
| P-0060 | 0.24 | 2.16 |
| P-0054 | 6.82E−03 | 19.8 |
| P-0055 | 1.24E−03 | 0.961 |
| P-0057 | 2.15E−03 | 1.7 |
| P-0056 | 0.602 | 0.647 |
| P-0058 | 1.89E−03 | 1.66E−03 |
| P-0072 | 1.26E−03 | 1.12E−02 |

### Epitope Mapping

To identify the approximate binding epitopes of the VHH antibodies, truncated ectodomains were expressed as fusion polypeptides with an AviTag. Additionally, biotin is added to the protein during expression to allow capture of the material onto Streptavidin-coated (SA) biosensors. SA biosensors (Sartorius) were incubated with one of the truncated ectodomains. Full length CD117 ectodomain was used as a control. The results show that all but P12 do not bind Domain 1. Except for P29 and P37, all others bind Domains 1-2 and Domains 1-3. Except for P37, all others bind Domains 1-4.

### Competition Studies Using SCF

VHHs were assessed to see whether they compete with Stem Cell Factor (SCF) for a binding location with CD117, using the biolayer interferometry method disclosed herein. Competition was most easily visualized when SCF bound to the CD117 ectodomain first, as it had a stable interaction with a slow off-rate relative to the VHH antibodies.

Two VHHs, P-0035 and P-0037 showed completion with SCF when SCF bound first. Some interference between VHHs and SCF was also observed when the VHHs bound to the CD117 ectodomain first. 5 VHHs P-0010, P-0012, P-0031, P-0035 and P-0037, showed some potential to block SCF binding when they were allowed to bind to the CD117 Ectodomain first.

### Competition Between VHH Antibodies to CD117.

VHHs were also tested to see whether they compete with each other for binding to the CD117 ectodomain using Biolayer Interferometry (Octet). The results from this study show that 4 VHHs, P10, P27, P31 and P38, competed with each other for binding to CD117, indicating that they may have similar/overlapping binding epitopes (falling in the same bin), whereas the rest fall into different bins.

### Impact on Cell Function

The 9 top VHH antibodies (in bivalent form) were investigated for their impact on the growth of CD34⁺ HSCs and ligand-induced C-kit phosphorylation. Most of the tested VHH antibodies (e.g., P10, P31, and P38) showed no impact on CD34⁺ cell growth. Similarly, these VHH antibodies (e.g., P10, P31, and P38) also showed no impact on inhibition of ligand-induced C-kit pTyr phosphorylation.

Certain features of exemplary anti-CD117 VHH antibodies as examined in this example is provided in Table 5 below.

TABLE 5
Representative Features of Exemplary anti-CD117 VHH Antibodies

| | | | | Inhibition of | | |
|---|---|---|---|---|---|---|
| | | | Inhibits | ligand- | Binds both | CD34 |
| Antibody | Epitope | Binding | SCF | induced | Human and | Anti- |
| name | Bin | properties | binding? | cKIT pTyr | NHP CD117? | growth |
| P10/P53 | Bin 1 | Good | No | No | Yes | No |
| P38/P72 | Bin 1 | Good | No | No | Yes | No |
| P31/P55 | Bin 1 | Good | No | No | Yes | No |
| P27/P60 | Bin 1 | Good | No | No | Yes | No |
| P37/P58 | Bin 2 | Good | Some | Yes | Yes | Yes |

### Example 2: Conjugating Anti-CD117 VHH to Lipid Nanoparticles to Develop a CD117-Targeting Delivery Vehicle

This example describes the methods and assays used to conjugate an anti-CD117 VHH (using P10 as an example) to lipid nanoparticles to develop a delivery vehicle specific to CD117⁺ cells.

### (i) Conjugation of VHH to Azide for VHH Functionalization

An azide functional group was coupled to the C-terminal of P10 by sortase reaction, which would enable covalent bonding of P10 with the dibenzocyclooctyne-functionalized polyethylene glycol (DBCO-PEG) in lipid nanoparticles to attach the anti-CD117 VHH on the surface of the lipid nanoparticles for specific targeting of CD117⁺ cells.

The nucleotide sequence encoding P10 was cloned into a vector for expression and secretion from Expi293f cells according to manufacturer's protocol (ThermoFischer Scientific). The P10-containing polypeptide (amino acid sequence shown below) thus expressed includes a sortase-mediated target LPETG (SEQ ID NO: 92) sequence (Sort-Tag) and a poly-histidine tag (HIS-Tag) at the C-terminus.

(SEQ ID NO: 28)
MELGLSWVVLAALLQGVQAQVQLVESGGGLVQAGGSLRLSCVASGRTFSS
YAMGWFRQGPGKEREFAAAILSNGLTTYYADSVKGRFTISRDNAKDTVYL
QMNSLKPEDTANYYCAAAFTPEFRDGGIWDDASVDYWGQGTQVTVSSGSG
GSLPETGGHHHHHHHHEPEA

The N-terminus signal peptide is italicized, and the G/S linkers are italicized and underlined. The Sort-tag is in boldface, and the His-tag is in boldface and italicized.

The transfected Expi293f cells were maintained in a shaking incubator for about 4 days to allow the VHH antibody to be expressed and secreted into the media. The cell supernatants were harvested by centrifugation and filtered to remove cell debris. Standard protein analytics were performed to estimate supernatant protein expression and yield. The His-Tag proteins were purified from the supernatant using immobilized metal affinity chromatography (IMAC) and was further concentrated using Amicon® centrifugal filters (Millipore Sigma) prior to a desalting step using PD-10 columns (Cytiva).

To add an azide moiety to the VHH antibody, a Sortase-A mediated conjugation reaction using a GGG-Lysine-azide peptide substrate was performed. The reaction was setup based on a protocol using hyperactive mutant sortase A (Sortase A5) as per manufacturer's instructions (Active Motif, Inc.). The reaction mix was optimized to include CD117 VHH, the GGG-Lysine-azide peptide and Sortase A5 in a molar ratio of 1:100:1, with 5 mM calcium chloride spiked into the buffer for efficient modification in 1 hour. This process removed the Sort-Tag and the His-Tag from the CD117 VHH sequence, while the C-terminus was modified with the GGGK peptide to generate the VHH-azide. Any unmodified VHH was removed by passing the reaction mixture over an IMAC column.

The resulting azide-functionalized VHH was concentrated and desalted into a buffer that is compatible for the click chemistry reaction described below, following routine practice.

### (ii) Covalent Coupling of VHH-Azide to DBCO-PEG Conjugated LNPs

The anti-CD117 VHH-azide polypeptide described above was covalently coupled to DBCO-PEG, which is conjugated to lipid nanoparticles (LNPs) (DBCO-PEG-LNP). The LNPs were encapsulated with either GFP mRNA or a gene editing system containing SpCas9 mRNA and a gRNA.

Briefly, the VHH-azide and DBCO-PEG-LNP were mixed in 1:3 molar ratio, respectively in a reaction buffer. After incubation for 1 hour at 4° C., an aliquot was collected to analyze the extent of conjugation by SDS-PAGE. The remainder of sample was transferred to a 100 kDa Amicon® centrifugal filter (Millipore Sigma) and washed with reaction buffer to remove unconjugated VHH-azide. The sample was then concentrated to the desired volume and encapsulation efficiency of the LNP and the concentration of the payload RNA quantified using a ribogreen assay (Thermo Fischer Scientific).

(iii) Assessment of Cargo Delivery Efficiency

The VHH-LNPs prepared as disclosed herein were examined for efficiency of delivering the cargo carried thereby (GFP mRNA or the gene editing system noted above).

For examining GFP expression, CD34⁺ HSCs and Kasumi-1 cells were plated at 30,000 cells per well of a flat bottom 96-well plate in 50 μL of media. CD117 targeting VHH-LNPs containing GFP mRNA were serially diluted based on the RNA amount (obtained from ribogreen assay) in media and 50 μL of each VHH-LNP dilution was added to individual wells containing the cells. The cells were incubated at 37° C. for 20 hours and then analyzed via flow cytometry for GFP expression and viability. As shown in FIGS. 2A-2B, VHH-LNPs successfully delivered GFP mRNA to both HSCs and Kasumi-1 cells, leading to GFP expressions in both cells, while only low fluorescence signals were observed in both types of cells treated with LNPs.

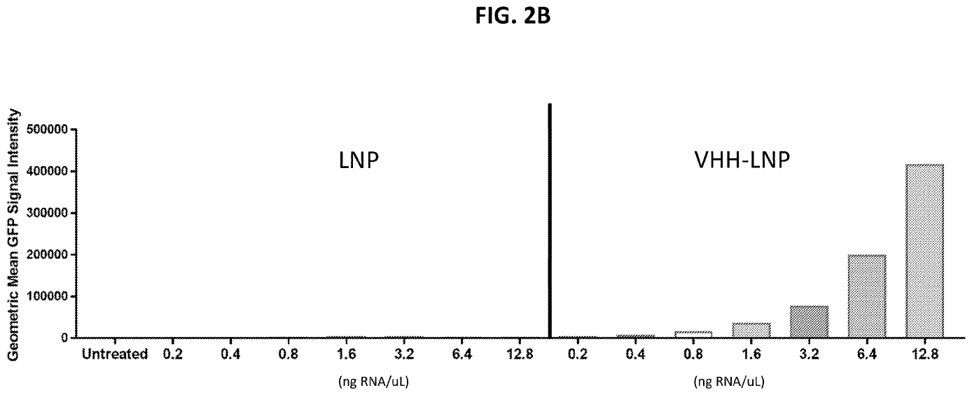

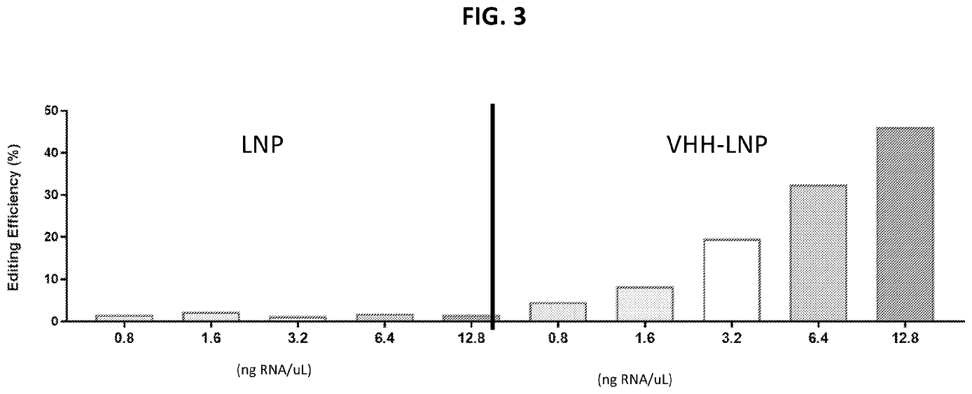

For examining editing levels, CD34⁺ and Kasumi-1 cells were plated at 30,000 cells per well of a flat bottom 96-well plate in 50 μL of media. CD117-targeting VHH-LNPs containing CCR5 gRNA TB7 and SpCas9 mRNA were serially diluted based on the RNA amount (obtained from the ribogreen assay) in media and 50 μL of each VHH-LNP dilution was added to individual wells containing the cells. The cells were incubated at 37° C. for 72 hours, after which genomic DNA was isolated and used in a PCR for TIDE analysis. Efficient VHH dependent editing in Kasumi-1 cells was observed when using VHH-LNP but not LNP alone. FIG. 3.

Cell viability in the presence of LNPs, with or without VHH, was also examined.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. An antibody that binds CD117, comprising a single domain antibody fragment, which comprises:
(i) a complementarity determining region 1 (CDR1) set forth as GX₁X₂TX₃X₄X₅X₆X₇ (SEQ ID NO: 2), in which X₁ is D, G, H, R, or T; X₂ is T or absent; X₃ is F, L, S, or V; X₄ is G, S, or T; X₅ is I, N, S, T or Y; X₆ is D, V, or Y; and X₇ is A, F, P, S, V or W;
(ii) a complementarity determining region 2 (CDR2) set forth as X₁X₂X₃X₄X₅X₆X₇X₈, in which X₁ is I or V; X₂ is A, G, H, L, R, S, T, or V, X₃ is R, S, or W; X₄ is G, N, S, or Y; X₅ is A, G, L, or absent; X₆ is A, D, G, L, or S; X₇ is G, M, S, T, or V; and X₈ is A, L, or T; and
(iii) a complementarity determining region 3 (CDR3) set forth as:
(SEQ ID NO: 69)
(a) GRFHPIRVDTA;
(SEQ ID NO: 71)
(b) ASGSNWRLGAIDEY;
(SEQ ID NO: 73)
(c) GQHLSGLGGSAWSIEG;
(SEQ ID NO: 75)
(d) RQYVGSGSYYLKKEGGY;
(SEQ ID NO: 77)
(e) DSTGVYGTGYVSSRKGRY;
(SEQ ID NO: 79)
(f) AFTPEFRDGGIWDDASV;
(SEQ ID NO: 83)
(g) VRRRWLIWQEEEY;
(SEQ ID NO: 85)
(h) DQRGVPAYYSDYALY;
(SEQ ID NO: 86)
(i) DESFPAYYSDYALY;
(SEQ ID NO: 67)
(j) VLRTGM;
(SEQ ID NO: 80)
(k) SDSYFYASPHLY;
(SEQ ID NO: 81)
(l) SDTYFYASPHLY;
or
(SEQ ID NO: 84)
(m) RRGTILVVQEYEY;
wherein the single domain antibody fragment binds CD117.

2. The antibody of claim 1, wherein the CDR3 is set forth as any one of (a)-(i).

3. The antibody of claim 1, wherein the CDR1 is set forth as:
(SEQ ID NO: 8)
(a) GRTTFSTYW;
(SEQ ID NO: 11)
(b) GGTFSIYP;
(SEQ ID NO: 14)
(c) GRTLSNYF;
(SEQ ID NO: 17)
(d) GRTFSSYA;
(SEQ ID NO: 20)
(e) GHTFSNYA;
(SEQ ID NO: 30)
(f) GDTFSSYS;
(SEQ ID NO: 36)
(g) GRTSGSYV;
or
(SEQ ID NO: 37)
(h) GRTFTYDA.

4. The antibody of claim 1, wherein the CDR2 is set forth as:
(SEQ ID NO: 43)
(a) ISWSAGMA;
(SEQ ID NO: 45)
(b) IGWSASGT;
(SEQ ID NO: 47)
(c) IHWSLGST;
(SEQ ID NO: 49)
(d) ITSSGLVA;
(SEQ ID NO: 51)
(e) ISWSGGST;
(SEQ ID NO: 53)
(f) ILSNGLTT;
(SEQ ID NO: 59)
(g) IRWSGGTT;
or
(SEQ ID NO: 63)
(h) ISWSAGMT.

5. (canceled)

6. The antibody of claim 1, wherein the single domain antibody fragment is a heavy chain variable domain antibody (VHH).

7. The antibody of claim 6, wherein the single domain antibody fragment has the structure of, from N-terminus to C-terminus, framework (FR) 1 (FR1)-CDR1-FR2-CDR2-FR3-CDR3-FR4, and wherein:
(a) the FR1 is set forth as X₁VQLVESGGGLVX₂AGX₃SLRLSCX₄X₅S (SEQ ID NO: 1), in which X₁ is E or Q; X₂ is Q or R; X₃ is G or D; X₄ is A, T, or V; and X₅ is A, G, or V;
(b) the FR2 is set forth as X₁X₂WX₃RQX₄PGKX₅REX₆VX₇X₈ (SEQ ID NO: 3), in which X₁ is L, M, R, or V; X₂ is A, G, or H; X₃ is F, L, or Y; X₄ is A or G; X₅ is E, N, Q, or R; X₆ is F or L; X₇ is A, G, or S; and X₈ is A, G or S;
(c) the FR3 is set forth as X₁YX₂DSX₃X₄GRFTISRDX₅X₆X₇X₈TVYLX₉MX₁₀SLKPEDTAZX₁₁YYCAA (SEQ ID NO: 40), in which X₁ is L, N, or Y; X₂ is A, G, L, P, or Q; X₃ is M, V, or absent; X₄ is E or K; X₅ is G, K, or N; X₆ is A, G, T, or V; X₇ is E, K, or R; X₈ is D, N, or S; X₉ is H, Q or R; X₁₀ is D, N, or S; and X₁₁ is N, T, or V; and
(d) the FR4 is set forth as X₁X₂WX₃QGTX₄VTVSS (SEQ ID NO: 66) in which X₁ is D, E, L, R, or T; X₂ is D, S, or Y; X₃ is G or A; and X₄ is L or Q.

8. The antibody of claim 7, wherein the FR1 comprises:
(SEQ ID NO: 7)
(a) EVQLVESGGGLVRAGGSLRLSCAAS;
(SEQ ID NO: 10)
(b) QVQLVESGGGLVQAGGSLRLSCAAS;
(SEQ ID NO: 13)
(c) QVQLVESGGGLVQAGDSLRLSCAVS;
(SEQ ID NO: 16)
(d) QVQLVESGGGLVQAGDSLRLSCAAS;
(SEQ ID NO: 19)
(e) QVHLVESGGGLVQAGGSLGLSCAAS;
(SEQ ID NO: 22)
(f) QVQLVESGGGLVQAGGSLRLSCVAS;
(SEQ ID NO: 29)
(g) QVQLVESGGGLVQAGGSLRLSCTAS;
or
(SEQ ID NO: 35)
(h) EVQLVESGGGLVQAGGSLRLSCAAS.

9. The antibody of claim 7, wherein the FR2 comprises:
(SEQ ID NO: 9)
(a) LGWFRQAPGKNREFVAA;
(SEQ ID NO: 12)
(b) VGWFRQAPGKQREFVAA;
(SEQ ID NO: 15)
(c) MAWLRQAPGKEREFVAA;
(SEQ ID NO: 18)
(d) MGWFRQAPGKEREFVAS;
(SEQ ID NO: 21)
(e) MGWFRQAPGKEREFVAA;
(SEQ ID NO: 23)
(f) MGWFRQGPGKEREFAAA;
(SEQ ID NO: 31)
(g) MGWFRQGPGKEREFVGG;
or
(SEQ ID NO: 38)
(h)RAWFRQAPGKEREFVAA.

10. The antibody of claim 7, wherein the FR3 comprises:
(SEQ ID NO 44)
(a) YYQDSKGRFTISRDNTKNTAYLQMNSLQPEDTAVYYCAA;
(SEQ ID NO: 46)
(b) YYGDSVEGRFTVSRDNARSTVYLRMSSLKPDDTAVYYCAA;
(SEQ ID NO: 48)
(c) YYQDPVKGRFTISRDKAKNTVYLQMNTLKPEDTATYICAA;
(SEQ ID NO: 50)
(d) YYGDSVEARFTISRDNAKNTVYLQMDSLKPEDTAVYYCAA;
(SEQ ID NO: 52)
(e) LYADSVKGRFTISRDNGENTVYLQMNSLKPEDTAVYYCAL;
(SEQ ID NO: 54)
(f) YYADSVKGRFTISRDNAKDTVYLQMNSLKPEDTANYYCAA;
(SEQ ID NO: 60)
(g) YYADSVKGRFTISRDNAKNTVYLQMNSLKREDTAVYYCAA;
(SEQ ID NO: 56)
(h) YYADSVKGRFTISRDNAKNTVYLQMNSLKPEDTAVYYCAA
or
(SEQ ID NO: 65)
(i) YYPNSMKGRFTISRDNAKNTVYLQMNSLKPEDTAVYYCAA.

11. The antibody of claim 7, wherein the FR4 comprises:
(SEQ ID NO: 70)
(a) LYWAQGTQVTVSS;
(SEQ ID NO: 72)
(b) DSWGQGTQVTVSS;
(SEQ ID NO: 74)
(c) DYWGQGTQVTVSS;
(SEQ ID NO: 76)
(d) EYWGPGTQVTVSS;
(SEQ ID NO: 78)
(e) EYWGQGTLVTVSS;
(SEQ ID NO: 68)
(f) DYWGQGTLVTVSS;
or
(SEQ ID NO: 87)
(g) RYWGQGTLVTVSS.

12. (canceled)

13. (canceled)

14. The antibody of claim 1, wherein the single domain antibody fragment comprises a sortase recognition motif at the C-terminus.

15. (canceled)

16. The antibody of claim 1, wherein the antibody comprises a functional group attached to the single domain antibody fragment.

17. (canceled)

18. The antibody of claim 1, wherein the antibody further comprises an Fc fragment, which is linked to the C-terminus of the single domain antibody fragment.

19. (canceled)

20. A lipid nanoparticle conjugate, comprising (a) a lipid nanoparticle (LNP), and (b) an antibody that binds CD117 (anti-CD117 antibody), wherein the anti-CD117 antibody is attached on the surface of the LNP and wherein the anti-CD117 antibody is set forth in claim 1.

21. (canceled)

22. (canceled)

23. (canceled)

24. The lipid nanoparticle conjugate of claim 20, further comprising a cargo, wherein the cargo is a gene editing system, which comprises a nuclease or a nucleic acid encoding the nuclease.

25. (canceled)

26. (canceled)

27. A method for delivering a cargo to cells, the method comprising:
contacting a lipid nanoparticle conjugate of claim 24 with cells expressing CD117 (CD117⁺ cells) to allow for delivery of the cargo contained in the lipid nanoparticle conjugate to the CD117⁺ cells.

28.-36. (canceled)

37. A nucleic acid comprising a nucleotide sequence encoding an anti-CD117 antibody set forth in claim 1.

38. A vector comprising the nucleic acid of claim 37, optionally wherein the vector is an expression vector.

39. A host cell comprising the vector of claim 38.

40. (canceled)


SEQUENCE LISTING (128 sequences)

SEQ ID NO: 1 - protein, 25 aa, Synthetic construct
  SITE 1: note = E or Q
  SITE 13: note = Q or R
  SITE 16: note = G or D
  SITE 23: note = A, T, or V
  SITE 24: note = A, G, or V
XVQLVESGGGLVXAGXSLRLSCXXS

SEQ ID NO: 2 - skipped

SEQ ID NO: 3 - protein, 17 aa, Synthetic construct
  SITE 1: note = L, M, R, or V
  SITE 2: note = A, G, or H
  SITE 4: note = F, L, or Y
  SITE 7: note = A or G
  SITE 11: note = E, N, Q, or R
  SITE 14: note = F or L
  SITE 16: note = A, G, or S
  SITE 17: note = A, G or S
XXWXRQXPGKXREXVXX

SEQ ID NO: 4 - protein, 25 aa, Synthetic construct
EVQLVESGGGLVQAGGSLRLSCVAS

SEQ ID NO: 5 - protein, 8 aa, Synthetic construct
GTTSGIVA

SEQ ID NO: 6 - protein, 17 aa, Synthetic construct
MHWYRQAPGKRRELVAS

SEQ ID NO: 7 - protein, 25 aa, Synthetic construct
EVQLVESGGGLVRAGGSLRLSCAAS

SEQ ID NO: 8 - protein, 9 aa, Synthetic construct
GRTTFSTYW

SEQ ID NO: 9 - protein, 17 aa, Synthetic construct
LGWFRQAPGKNREFVAA

SEQ ID NO: 10 - protein, 25 aa, Synthetic construct
QVQLVESGGGLVQAGGSLRLSCAAS

SEQ ID NO: 11 - protein, 8 aa, Synthetic construct
GGTFSIYP

SEQ ID NO: 12 - protein, 17 aa, Synthetic construct
VGWFRQAPGKQREFVAA

SEQ ID NO: 13 - protein, 25 aa, Synthetic construct
QVQLVESGGGLVQAGDSLRLSCAVS

SEQ ID NO: 14 - protein, 8 aa, Synthetic construct
GRTLSNYF

SEQ ID NO: 15 - protein, 17 aa, Synthetic construct
MAWLRQAPGKEREFVAA

SEQ ID NO: 16 - protein, 25 aa, Synthetic construct
QVQLVESGGGLVQAGDSLRLSCAAS

SEQ ID NO: 17 - protein, 8 aa, Synthetic construct
GRTFSSYA

SEQ ID NO: 18 - protein, 17 aa, Synthetic construct
MGWFRQAPGKEREFVAS

SEQ ID NO: 19 - protein, 25 aa, Synthetic construct
QVHLVESGGGLVQAGGSLGLSCAAS

SEQ ID NO: 20 - protein, 8 aa, Synthetic construct
GHTFSNYA

SEQ ID NO: 21 - protein, 17 aa, Synthetic construct
MGWFRQAPGKEREFVAA

SEQ ID NO: 22 - protein, 25 aa, Synthetic construct
QVQLVESGGGLVQAGGSLRLSCVAS

SEQ ID NO: 23 - protein, 17 aa, Synthetic construct
MGWFRQGPGKEREFAAA

SEQ ID NO: 24 - protein, 25 aa, Synthetic construct
QVQLVESGGGLVQAGGSLRLSCAGS

SEQ ID NO: 25 - protein, 8 aa, Synthetic construct
GRTVSTYA

SEQ ID NO: 26 - protein, 17 aa, Synthetic construct
MGWFRQAPGKEREFVSA

SEQ ID NO: 27 - protein, 25 aa, Synthetic construct
QVQLVESGGGLVQAGGSLRLSCVGS

SEQ ID NO: 28 - protein, 17 aa, Synthetic construct
MGWFRQAPGKERELVAA

SEQ ID NO: 29 - protein, 25 aa, Synthetic construct
QVQLVESGGGLVQAGGSLRLSCTAS

SEQ ID NO: 30 - protein, 8 aa, Synthetic construct
GDTFSSYS

SEQ ID NO: 31 - protein, 17 aa, Synthetic construct
MGWFRQGPGKEREFVGG

SEQ ID NO: 32 - protein, 25 aa, Synthetic construct
QVQLVESGGGLVQAGGSLRLSCAAS

SEQ ID NO: 33 - protein, 8 aa, Synthetic construct
GRTFSSYV

SEQ ID NO: 34 - protein, 17 aa, Synthetic construct
MGWFRQAPGKEREFVAA

SEQ ID NO: 35 - protein, 25 aa, Synthetic construct
EVQLVESGGGLVQAGGSLRLSCAAS

SEQ ID NO: 36 - protein, 8 aa, Synthetic construct
GRTSGSYV

SEQ ID NO: 37 - protein, 8 aa, Synthetic construct
GRTFTYDA

SEQ ID NO: 38 - protein, 17 aa, Synthetic construct
RAWFRQAPGKEREFVAA

SEQ ID NO: 39 - skipped

SEQ ID NO: 40 - protein, 41 aa, Synthetic construct
  SITE 1: note = L, N, or Y
  SITE 3: note = A, G, L, P, or Q
  SITE 6: note = M, V, or absent
  SITE 7: note = E or K
  SITE 16: note = G, K, or N
  SITE 17: note = A, G, T, or V
  SITE 18: note = E, K, or R
  SITE 19: note = D, N, or S
  SITE 24: note = H, Q or R
  SITE 26: note = D, N, or S
  SITE 36: note = N, T or V
XYXDSXXGRFTISRDXXXXTVYLXMXSLKPEDTAZXYYCAA

SEQ ID NO: 41 - protein, 7 aa, Synthetic construct
VVRGASL

SEQ ID NO: 42 - protein, 40 aa, Synthetic construct
NYLDSVKGRFIISEDGARNTVYLQMNSLKPEDTAVYYCNA

SEQ ID NO: 43 - protein, 8 aa, Synthetic construct
ISWSAGMA

SEQ ID NO: 44 - protein, 39 aa, Synthetic construct
YYQDSKGRFTISRDNTKNTAYLQMNSLQPEDTAVYYCAA

SEQ ID NO: 45 - protein, 8 aa, Synthetic construct
IGWSASGT

SEQ ID NO: 46 - protein, 40 aa, Synthetic construct
YYGDSVEGRFTVSRDNARSTVYLRMSSLKPDDTAVYYCAA

SEQ ID NO: 47 - protein, 8 aa, Synthetic construct
IHWSLGST

SEQ ID NO: 48 - protein, 40 aa, Synthetic construct
YYQDPVKGRFTISRDKAKNTVYLQMNTLKPEDTATYICAA

SEQ ID NO: 49 - protein, 8 aa, Synthetic construct
ITSSGLVA

SEQ ID NO: 50 - protein, 40 aa, Synthetic construct
YYGDSVEARFTISRDNAKNTVYLQMDSLKPEDTAVYYCAA

SEQ ID NO: 51 - protein, 8 aa, Synthetic construct
ISWSGGST

SEQ ID NO: 52 - protein, 40 aa, Synthetic construct
LYADSVKGRFTISRDNGENTVYLQMNSLKPEDTAVYYCAL

SEQ ID NO: 53 - protein, 8 aa, Synthetic construct
ILSNGLTT

SEQ ID NO: 54 - protein, 40 aa, Synthetic construct
YYADSVKGRFTISRDNAKDTVYLQMNSLKPEDTANYYCAA

SEQ ID NO: 55 - protein, 8 aa, Synthetic construct
IARSGGGT

SEQ ID NO: 56 - protein, 40 aa, Synthetic construct
YYADSVKGRFTISRDNAKNTVYLQMNSLKPEDTAVYYCAA

SEQ ID NO: 57 - protein, 8 aa, Synthetic construct
IGRSGGST

SEQ ID NO: 58 - protein, 40 aa, Synthetic construct
YYADSVKGRFTISRDNVRNTVYLQMNSLKPEDAAVYYCAA

SEQ ID NO: 59 - protein, 8 aa, Synthetic construct
IRWSGGTT

SEQ ID NO: 60 - protein, 40 aa, Synthetic construct
YYADSVKGRFTISRDNAKNTVYLQMNSLKREDTAVYYCAA

SEQ ID NO: 61 - protein, 8 aa, Synthetic construct
ISWYGDST

SEQ ID NO: 62 - protein, 40 aa, Synthetic construct
YYADSVKGRFTISRDNAKNTVYLHMNSLKPEDTAVYYCAA

SEQ ID NO: 63 - protein, 8 aa, Synthetic construct
ISWSAGMT

SEQ ID NO: 64 - protein, 40 aa, Synthetic construct; identical to SEQ ID NO: 56

SEQ ID NO: 65 - protein, 40 aa, Synthetic construct
YYPNSMKGRFTISRDNAKNTVYLQMNSLKPEDTAVYYCAA

SEQ ID NO: 66 - protein, 13 aa, Synthetic construct
  SITE 1: note = D, E, L, R, or T
  SITE 2: note = D, S, or Y
  SITE 4: note = G or A
  SITE 8: note = L or Q
XXWXQGTXVTVSS

SEQ ID NO: 67 - protein, 6 aa, Synthetic construct
VLRTGM

SEQ ID NO: 68 - protein, 13 aa, Synthetic construct
DYWGQGTLVTVSS

SEQ ID NO: 69 - protein, 11 aa, Synthetic construct
GRFHPIRVDTA

SEQ ID NO: 70 - protein, 13 aa, Synthetic construct
LYWAQGTQVTVSS

SEQ ID NO: 71 - protein, 14 aa, Synthetic construct
ASGSNWRLGAIDEY

SEQ ID NO: 72 - protein, 13 aa, Synthetic construct
DSWGQGTQVTVSS

SEQ ID NO: 73 - protein, 16 aa, Synthetic construct
GQHLSGLGGSAWSIEG

SEQ ID NO: 74 - protein, 13 aa, Synthetic construct
DYWGQGTQVTVSS

SEQ ID NO: 75 - protein, 17 aa, Synthetic construct
RQYVGSGSYYLKKEGGY

SEQ ID NO: 76 - protein, 13 aa, Synthetic construct
EYWGPGTQVTVSS

SEQ ID NO: 77 - protein, 18 aa, Synthetic construct
DSTGVYGTGYVSSRKGRY

SEQ ID NO: 78 - protein, 13 aa, Synthetic construct
EYWGQGTLVTVSS

SEQ ID NO: 79 - protein, 17 aa, Synthetic construct
AFTPEFRDGGIWDDASV

SEQ ID NO: 80 - protein, 12 aa, Synthetic construct
SDSYFYASPHLY

SEQ ID NO: 81 - protein, 12 aa, Synthetic construct
SDTYFYASPHLY

SEQ ID NO: 82 - protein, 13 aa, Synthetic construct
TDWGQGTQVTVSS

SEQ ID NO: 83 - protein, 13 aa, Synthetic construct
VRRRWLIWQEEEY

SEQ ID NO: 84 - protein, 13 aa, Synthetic construct
RRGTILVVQEYEY

SEQ ID NO: 85 - protein, 15 aa, Synthetic construct
DQRGVPAYYSDYALY

SEQ ID NO: 86 - protein, 14 aa, Synthetic construct
DESFPAYYSDYALY

SEQ ID NO: 87 - protein, 13 aa, Synthetic construct
RYWGQGTLVTVSS

SEQ ID NO: 88 - protein, 5 aa, Synthetic construct
LPXTG

SEQ ID NO: 89 - protein, 6 aa, Synthetic construct
LPETGG

SEQ ID NO: 90 - protein, 8 aa, Synthetic construct
LPXTGGGK

SEQ ID NO: 91 - protein, 8 aa, Synthetic construct
LPETGGGK

SEQ ID NO: 92 - protein, 5 aa, Synthetic construct
LPETG

SEQ ID NO: 93 - protein, 4 aa, Synthetic construct
GGGK

SEQ ID NO: 94 - skipped

SEQ ID NO: 95 - protein, 384 aa, Synthetic construct
CAAGTGCAGCTGGTGGAATCCGGAGGAGGACTGGTGCAAGCCGGCGGATCTCTGAGACTGAGCTGTGTGGCCAGCGGAAGAACCTTCAGCAGCTACGCCATGGGCTGGTTTAGACAAGGCCCCGGCAAAGAGAGGGAGTTTGCCGCCGCCATTCTGTCCAACGGACTGACAACCTACTACGCCGACAGCGTGAAGGGAAGATTCACCATCAGCAGAGACAACGCCAAGGACACCGTGTATCTGCAGATGAACTCTCTGAAGCCCGAGGACACCGCCAACTACTACTGCGCCGCCGCCTTTACCCCCGAGTTTAGGGACGGCGGCATCTGGGACGATGCCAGCGTGGACTACTGGGGCCAAGGCACACAAGTGACCGTGTCCAGC

SEQ ID NO: 96 - protein, 378 aa, Synthetic construct
GAGGTGCAGCTGGTGGAGAGCGGAGGAGGACTGGTGCAAGCCGGAGGCTCTCTGAGACTGAGCTGTGCCGCCAGCGGAAGAACCAGCGGCAGCTACGTGATGGGCTGGTTCAGACAAGCCCCCGGCAAGGAGAGGGAATTCGTGGCCGCCATTAGCTGGAGCGCCGGCATGACCTACTACGCCGACAGCGTGAAGGGAAGATTCACCATCTCTAGAGACAACGCCAAGAACACCGTGTATCTGCAGATGAACTCTCTGAAGCCCGAGGACACCGCCGTGTACTACTGTGCCGCCGATCAGAGAGGCGTGCCCGCCTACTACTCCGACTACGCTCTGTACGACTACTGGGGCCAAGGCACCCAAGTGACCGTGAGCAGC

SEQ ID NO: 97 - protein, 384 aa, Synthetic construct
CAAGTGCAGCTGGTGGAAAGCGGCGGAGGACTGGTGCAAGCCGGAGACTCTCTGAGACTGAGCTGCGCCGCCAGCGGCAGAACCTTCAGCAGCTACGCCATGGGCTGGTTCAGACAAGCCCCCGGCAAGGAGAGAGAGTTCGTGGCCAGCATCACCAGCAGCGGACTGGTGGCCTACTACGGAGACTCCGTGGAGGCCAGATTCACCATCTCTAGAGACAACGCCAAGAATACCGTGTATCTGCAGATGGACTCTCTGAAGCCCGAGGACACCGCTGTGTACTACTGTGCTGCTAGACAGTATGTGGGAAGCGGCTCCTACTATCTGAAGAAGGAGGGCGGCTACGAGTACTGGGGACCCGGCACCCAAGTGACCGTGTCCAGC

SEQ ID NO: 98 - protein, 58 aa, Synthetic construct
EVQLVESGGGLVQAGGSLRLSCVASGTTSGIVAMHWYRQAPGKRRELVASVVRGASLN

SEQ ID NO: 99 - protein, 60 aa, Synthetic construct
EVQLVESGGGLVRAGGSLRLSCAASGRTTFSTYWLGWFRQAPGKNREFVAAISWSAGMAY

SEQ ID NO: 100 - protein, 59 aa, Synthetic construct
QVQLVESGGGLVQAGGSLRLSCAASGGTFSIYPVGWFRQAPGKQREFVAAIGWSASGTY

SEQ ID NO: 101 - protein, 59 aa, Synthetic construct
QVQLVESGGGLVQAGDSLRLSCAVSGRTLSNYFMAWLRQAPGKEREFVAAIHWSLGSTY

SEQ ID NO: 102 - protein, 59 aa, Synthetic construct
QVQLVESGGGLVQAGDSLRLSCAASGRTFSSYAMGWFRQAPGKEREFVASITSSGLVAY

SEQ ID NO: 103 - protein, 59 aa, Synthetic construct
QVHLVESGGGLVQAGGSLGLSCAASGHTFSNYAMGWFRQAPGKEREFVAAISWSGGSTL

SEQ ID NO: 104 - protein, 59 aa, Synthetic construct
QVQLVESGGGLVQAGGSLRLSCVASGRTFSSYAMGWFRQGPGKEREFAAAILSNGLTTY

SEQ ID NO: 105 - protein, 59 aa, Synthetic construct
QVQLVESGGGLVQAGGSLRLSCAGSGRTVSTYAMGWFRQAPGKEREFVSAIARSGGGTY

SEQ ID NO: 106 - protein, 59 aa, Synthetic construct
QVQLVESGGGLVQAGGSLRLSCVGSGRTVSTYAMGWFRQAPGKERELVAAIGRSGGSTY

SEQ ID NO: 107 - protein, 59 aa, Synthetic construct
QVQLVESGGGLVQAGGSLRLSCTASGDTFSSYSMGWFRQGPGKEREFVGGIRWSGGTTY

SEQ ID NO: 108 - protein, 59 aa, Synthetic construct
QVQLVESGGGLVQAGGSLRLSCAASGRTFSSYVMGWFRQAPGKEREFVAAISWYGDSTY

SEQ ID NO: 109 - protein, 59 aa, Synthetic construct
EVQLVESGGGLVQAGGSLRLSCAASGRTSGSYVMGWFRQAPGKEREFVAAISWSAGMTY

SEQ ID NO: 110 - protein, 59 aa, Synthetic construct
EVQLVESGGGLVRAGGSLRLSCAASGRTFTYDARAWFRQAPGKEREFVAAISWSGGSTY

SEQ ID NO: 111 - protein, 48 aa, Synthetic construct
YLDSVKGRFIISEDGARNTVYLQMNSLKPEDTAVYYCNAVLRTGMDYW

SEQ ID NO: 112 - protein, 52 aa, Synthetic construct
YQDSKGRFTISRDNTKNTAYLQMNSLQPEDTAVYYCAAGRFHPIRVDTALYW

SEQ ID NO: 113 - protein, 56 aa, Synthetic construct
YGDSVEGRFTVSRDNARSTVYLRMSSLKPDDTAVYYCAAASGSNWRLGAIDEYDSW

SEQ ID NO: 114 - protein, 58 aa, Synthetic construct
YQDPVKGRFTISRDKAKNTVYLQMNTLKPEDTATYICAAGQHLSGLGGSAWSIEGDYW

SEQ ID NO: 115 - protein, 59 aa, Synthetic construct
YGDSVEARFTISRDNAKNTVYLQMDSLKPEDTAVYYCAARQYVGSGSYYLKKEGGYEYW

SEQ ID NO: 116 - protein, 60 aa, Synthetic construct
YADSVKGRFTISRDNGENTVYLQMNSLKPEDTAVYYCALDSTGVYGTGYVSSRKGRYEYW

SEQ ID NO: 117 - protein, 59 aa, Synthetic construct
YADSVKGRFTISRDNAKDTVYLQMNSLKPEDTANYYCAAAFTPEFRDGGIWDDASVDYW

SEQ ID NO: 118 - protein, 54 aa, Synthetic construct
YADSVKGRFTISRDNAKNTVYLQMNSLKPEDTAVYYCAASDSYFYASPHLYDYW

SEQ ID NO: 119 - protein, 54 aa, Synthetic construct
YADSVKGRFTISRDNVRNTVYLQMNSLKPEDAAVYYCAASDTYFYASPHLYTDW

SEQ ID NO: 120 - protein, 55 aa, Synthetic construct
YADSVKGRFTISRDNAKNTVYLQMNSLKREDTAVYYCAAVRRRWLIWQEEEYDYW

SEQ ID NO: 121 - protein, 55 aa, Synthetic construct
YADSVKGRFTISRDNAKNTVYLHMNSLKPEDTAVYYCAARRGTILVVQEYEYDYW

SEQ ID NO: 122 - protein, 57 aa, Synthetic construct
YADSVKGRFTISRDNAKNTVYLQMNSLKPEDTAVYYCAADQRGVPAYYSDYALYDYW

SEQ ID NO: 123 - protein, 56 aa, Synthetic construct
YPNSMKGRFTISRDNAKNTVYLQMNSLKPEDTAVYYCAADESFPAYYSDYALYRYW

SEQ ID NO: 124 - protein, 10 aa, Synthetic construct
GQGTLVTVSS

SEQ ID NO: 125 - protein, 10 aa, Synthetic construct
AQGTQVTVSS

SEQ ID NO: 126 - protein, 10 aa, Synthetic construct
GQGTQVTVSS

SEQ ID NO: 127 - protein, 10 aa, Synthetic construct
GPGTQVTVSS

SEQ ID NO: 128 - protein, 170 aa, Synthetic construct
MELGLSWVVLAALLQGVQAQVQLVESGGGLVQAGGSLRLSCVASGRTFSSYAMGWFRQGPGKEREFAAAILSNGLTTYYADSVKGRFTISRDNAKDTVYLQMNSLKPEDTANYYCAAAFTPEFRDGGIWDDASVDYWGQGTQVTVSSGSGGSLPETGGHHHHHHHHEPEA